(19) United States
(12) Patent Application Publication
(10) Pub. No.: US 2009/0209508 A1
(43) Pub. Date: Aug. 20, 2009

(54) Compounds for Photochemotherapy

(75) Inventors: Norbert Lange, Nyon (CH); Marino A. Campo, Nyon (CH)

Correspondence Address: FULBRIGHT & JAWORSKI L.L.P., 600 CONGRESS AVE., SUITE 2400, AUSTIN, TX, 78701, US

(73) Assignee: UNIVERSITE DE GENEVE, Geneve 4 (CH)

(21) Appl. No.: 11/914,446

(22) PCT Filed: May 15, 2006
(86) PCT No.: PCT/IB06/03547

§ 371 Date: Nov. 20, 2008

(30) Foreign Application Priority Data
May 16, 2005 (US) 60/681244

(51) Int. Cl. A61K 41/00; A61K 31/409; A61K 31/555; A61P 35/00; A61P 31/00

(52) U.S. Cl. 514/185; 514/410

(57) ABSTRACT

Enzyme-activatable photosensitizing polymer conjugates are disclosed for photochemotherapeutic treatment of human diseases and disorders, bacteriologic or virologic indications, cosmetic applications and other pathologic situations. These polymer conjugates may comprise a polymer carrier, a photosensitizer, a quencher, a targeting molecule and/or a biocompatibilizing unit. These macromolecular conjugates may be designed to guide to the target tissue a photosensitizing agent in an inactive, non-phototoxic form. However, upon entering the target environment, in which certain enzymes are presently active, the conjugate may release its photosensitizers in its fully active form, resulting in a highly localized activation of the photoactive agent. Also described here are methods, compositions and kits for the preparation and testing of such photochemotherapcutic conjugates.

This application claims the benefit of U.S. Provisional Application Ser. No. 60/681,244 filed May 16, 2005, the entire contents and disclosures of which are specifically incorporated by reference herein without disclaimer.

### BACKGROUND OF THE INVENTION

1. Field of the Invention

The present invention relates generally to the fields of chemistry, pharmacology, and molecular biology. More particularly, it concerns compositions comprising photosensitizers and uses thereof.

2. Description of Related Art

Photochemotherapy (PCT) is a modality for the treatment of human diseases and disorders, bacteriological indications, and other pathological situations. Furthermore, PCT has also been used for cosmetic purposes such as hair removal, the treatment of acne, and skin rejuvenation. PCT is based on the topical or systemic application of a photosensitizing agent or a precursor or prodrug thereof, which ideally accumulates with some degree of selectivity in the target tissue (Pech et al., 2001; De Rosa, 2000; Bressler and Bressler, 2000; Sheski and Mathur, 2000; His et al., 1999; Biel, 1998; Wainwright, 1998; Dougherty et al., 1998; Nseyo, 1992; Spitzer and Krumholz, 1991), followed by irradiation of the photosensitizing agent with light of an appropriate wavelength, which generates reactive oxygen species due to the interaction of the thus excited photosensitizer with oxygen, leading to tissue damage and destruction of the irradiated areas. It is important to note that only the presence of each of the tluee components involved in the PCT process, light, photosensitizer, and oxygen, results in the desired therapeutic process.

Photosensitizing agents have significant limitations that limit their clinical potential. Historically, the first photosensitizing agent, which was used for the treatment of cancer, is hematoporphyrin derivative (HpD) (Gomer et al., 1979), a complex mixture of porphyrin dimers and oligomers involving ether, ester, and other linkages. Although HpD and its commercial, purified variants have been used extensively in experimental clinical work, these first generation photosensitizers have at least three important disadvantages. Firstly, they lack selectivity for the target tissue and cause prolonged skin photosensitization due to slow body clearance. Secondly, the absorption in the red wavelength region, where light penetration into the tissue is favored, is relatively weak. Thirdly, they are ill-defined mixtures that give difficult to reproduce results.

Due to the drawbacks of conventional photochemotherapeutic agents, research has focused on the development of more potent photosensitizing agents, having better properties with respect to an effective PCT treatment (Stemberg and Dolphin, 1993). However, despite considerable research efforts in this field, the ideal photosensitizer has not been found yet. In view of the huge diversity of human neoplastic and non-neoplastic disorders and abnormalities this fact is not surprising. To begin with, these photosensitizers lack the required selectivity for target tissue and also exhibit dark toxicity. In addition, they induce skin photosensitization for long periods of time, due to their slow clearance from the body Thus, research in the field of PCT has switched to the development of more selective, targeting photosensitizers, in part based on the recent progress made in molecular biology and biochemistry.

Following concepts of controlled drug delivery, one approach is based on the covalent coupling of a photosensitizing moiety to a carrier unit that specifically binds to cellular functions, found in abundance in cells associated with the corresponding disease (for review see Lange et al., 2002 and references therein). Typical examples for such targets include antigens (Vrouenraets et al., 2001; Vrouenraets et al., 2000; Del Governatore et al., 2000), cell surface receptors (Hamblin et al., 2000; James et al., 1999; Nagae et al., 1998), and cell adhesion molecules. Since the characteristics of tumor selectivity are no longer dominated by the pharmacokinetic properties of the photosensitizing agent itself, now its properties can be adapted with respect to tissue optics, singlet oxygen quantum yield and the clinical situation. Very recently, Neri et al. (Bircher et al., 1999a; Bircher et al., 1999b) have used single chain antibody fragments (scFv) coupled to the photosensitizer Tin(IV) chlorin e6 to induce selective photothrombosis in experimental animal models used for angiogenic research.

However, most of these new PCT targeting agents address cellular functions associated with angiogenesis, i.e. in the case of cancer in a somewhat advanced stage of its development. Other targeting photosensitizers coupled to antibodies (Vrouenraets et al., 2001; Vrouenraets et al., 2000; Del Governatore et al., 2000) as specific carrier moieties have unfavorable pharmacokinetic properties, may provoke immune responses, or lack penetration into the tumor mass. Furthermore, due to the short lifetime of reactive oxygen species (ROS) in biological tissue and consequently their limited radius of action, targeting of functions expressed on the cell surface might significantly reduce the phototoxic efficacy of the targeting photosensitizer (Rosenkranz et al., 2000). From this point of view, using “cargo” type receptors that serve to deliver metabolic substrates to the target appears more promising. However, this class of receptors (Akhlynina et al., 1995), including the insulin receptor the low density lipoprotein receptor (Haimovici et al., 1997), and the transferrin receptor (Hamblin and Newman, 1994) are often not sufficiently specific and camiot be used for a wide range of diseases. WO2004/004769 involves the use of photosensitizers in molecular beacons.

Contrary to the targeting of cell receptors, enzymatic targeting offers a more promising approach to treat a wide variety of diseases such as cancer. It is well known that many neoplastic and non-neoplastic pathological conditions can be linked directly or indirectly to abnormal enzymatic activity (see Table 1). Considerable efforts have been made to develop treatments based on enzyme inhibitors to manage, treat or cure some of these disorders (e.g., WO 2005007631; Coussens et al., 2002), but with only limited success. Besides toxicity issues, the problem with such treatments arise from acquired resistance to the inhibitors through either mutations (Novartis Gleevec) (Hochhaus and LaRosse, 2004) or multidrug cellular efflux systems.

TABLE 1
Enzymes that are related to some pathological conditions.

| Enzyme | Disease or pathology |
|---|---|
| Fructokinase, Fructose 1,6-diphosphate | Disorders in carbohydrate |
| aldolase B, Fructose 1,6-diphosphatase, | metabolism |
| Glucose 6-phosphatase, Glucose 6- | |
| phosphate translocase, alpha-Glucosidase | |
| (lysosomal), Amylo-1,6-glucosidase, | |
| Amylo-1,4:1,6-glucantransferase, | |
| Phosphorylase b-kinase, | |
| Phosphofructokinase, Glycogen synthase, | |
| Phosphoglycerate kinase, | |
| Phosphoglycerate mutase, Lactate | |
| dehydrogenase, Glucose phosphate | |
| isomerase, Galactose-1-phosphate | |
| uridyltransferase, Galactokinase, Uridine | |
| diphosphate galactose 4-epimerase, L- | |
| xylulose reductase, | |
| Phenylalanine hydroxylase, | Disorders in amino acid |
| Dihydropteridine reductase, Guanosine | metabolism |
| triphosphate cyclohydrolase, 6-Pyruvoyl | |
| tetrahydropterin synthase, | |
| Fumarylacetoacetate hydrolyase, | |
| Maleylacetoacetate isomerase, Tyrosine | |
| aminotransferase, Urocanase, Histidase, | |
| Proline oxidase, DELTA.-Pyrrolidine-5- | |
| carboxylate dehydrogenase, 4-Hydroxy- | |
| L-proline-oxidase, Peptidase D, | |
| Omithine-delta-aminotransferase, | |
| Carbamyl phosphate synthase, Omithine | |
| transcarbamylase, Argininosuccinic acid | |
| synthase, Argininosuccinic acid synthase, | |
| Arginase, alpha-Aminoadipic | |
| semialdehyde synthase, Cysthathionine | |
| beta-synthase, alpha.-Cystathionase, | |
| Methionine adenosyltransferase, | |
| Sarcosine dehydrogenase, | |
| Dihydropyrimidine dehydrogenase, beta- | |
| Alanine-pyruvate transaminase, R-beta- | |
| Aminoisobutyrate-pyruvate transaminase, | |
| Glutamic acid decarboxylase, GABA- | |
| alpha-Ketoglutarate transaminase, | |
| Succinic semialdehyde dehydrogenase, | |
| Carnosinase. | |
| Homogentisic acid oxidase, Isovaleryl- | Disorders in metabolism of |
| CoA dehydrogenase, 3- | organic acids |
| Methylcrotononyl-CoA carboxylase, 3- | |
| Methylglutaconyl-CoA hydratase, | |
| Mevalonate kinase, 2-Methylacetoacetyl- | |
| CoA thiolase, 3-Hydroxyisobutyryl-CoA | |
| deacylase, Propionyl-CoA carboxylase, | |
| Methylmalonyl-CoA mutase, | |
| ATP: Cobalamin adenosyltransferase, | |
| Glutaryl-CoA dehydrogenase, 2- | |
| Ketoadipic acid dehydrogenase, | |
| Glutathione synthetase, 5-Xoprolinase, | |
| gamma-Glutamylcysteine synthetase, | |
| delta-Glutamyl transpeptidase, | |
| Cytochrome oxidase, Fumarase, Pyruvate | |
| carboxylase, Long-chain acyl-CoA | |
| dehydrogenase, Medium-chain acyl-CoA | |
| dehydrogenase, Short-chain acyl-CoA | |
| dehydrogenase, Electron transfer | |
| flavoprotein: ubiquinone oxidoreductase, | |
| Alanine: glyoxylate aminotransferase, D- | |
| Glycerate dehydrogenase, Glycerol | |
| kinase. | |
| PP-Ribose-P synthetase, Hypoxanthine- | Disorders in metabolism of |
| guanine phosphoribosyltransferase, | purines and pyrimidines |
| Adenine phosphoribosyltransferase, | |
| Adenosine deaminase, Purine nucleoside | |
| phosphorylase, Myoadenylate deaminase, | |
| Xanthine dehydrogenase, UMP synthase, | |
| Pyrimidine 5′nucleotidase, | |
| Dihydropyrimidine dehydrogenase, | |
| Lipoprotein lipase, Lecithin: cholesterol | Disorders of lipid |
| acyltransferase, 26-hydroxylase | metabolism |
| (cholesterol), | |
| delta-Aminolevulinic acid dehydratase, | Disorders in metabolism |
| Porphobilinogen deaminase, | of porphyrins and heme |
| Uroporphyrinogen cosynthase, | |
| Uroporphyrinogen decarboxylase, | |
| Coproporphyrinogen oxidase, | |
| Protoporphyrinogen oxidase, | |
| Ferrochelatase, Bilirubin UDPglucuronyl | |
| transferase, Phytanic acid alpha- | |
| hydroxylase, Catalase. | |
| alpha-L-iduronidase, Iduronate sulfatase, | Disorders of lysosomal |
| Heparan-N-sulfatase, alpha-N- | enzymes |
| acetylglucosaminidase, Acetyl-CoA- | |
| .alpha.-glucosaminide acetyltransferase, | |
| Acetylglucosamine 6-sulfatase, Galactose | |
| 6-sulfatase, beta-Galactosidase, N- | |
| Acetylgalactosamine 4-sulfatase, beta- | |
| Glucuronidase, UDP: N- | |
| Acetylglucosamine: lysosomal enzyme | |
| N-acetylglucosaminyl-1- | |
| phosphotransferase, alpha-Mannosidase, | |
| alpha-Neuraminidase, | |
| Aspartylglucosaminidase, alpha-L- | |
| Fucosidase, Acid lipase, Acid | |
| ceramidase, Sphingomyelinase, | |
| Glucocerebrosidase, | |
| Galactosylceramidase, Steroid sulfatase, | |
| Arylsulfatase, alpha-Galactosidase, alpha- | |
| N-Acetylgalactosaminidase, Acid beta- | |
| galactosidase, beta.-Hexosaminidase. | |
| Steroid 21-hydroxylase, Steroid 5-alpha- | Disorders in metabolism of |
| reductase, 3-beta-Hydroxysteroid | hormones |
| sulfatase, 25(OH)D₃-1-alpha- | |
| hydroxylase. | |
| Methylene tetrahydrofolate reductase, | Disorders in metabolism of |
| Glutamate formiminotransferase, | vitamins |
| Holocarboxylase synthetase, Biotinidase. | |
| Cytochrome b₅ reductase, Pyruvate | Disorders of blood |
| kinase, Hexokinase, Glucosephosphate | |
| isomerase, Aldolase, Triosephosphate | |
| isomerase, Phosphoglycerate kinase, | |
| 2,3-Diphosphoglyceromutase, | |
| 6-Phosphogluconate dehydrogenase, | |
| Gluthathione peroxidase, Gluthathione | |
| reductase, Gluthathione synthetase, | |
| gamma-Glutamylcysteine synthetase, | |
| Adenosine deaminase, Pyrimidine | Disorders of the immune |
| nucelotidase, Myeloperoxidase, NADPH | system |
| oxidase. | |
| Lysyl hydroxylase, Collagenase, Alkaline | Disorders of connective |
| phosphatase, Carbonic anhydrase. | tissues |
| Tyrosinase. | Disorders of the skin |
| Lactase, Trehalase. | Disorders of the digestive |
| | system |
| Cathepsin D, Cathepsin B, Cathepsin H, | Neoplastic disorders |
| Prostate specific antigen (PSA), Matrix | |
| metalloproteinases, CMV protease | |
| Matrix metalloproteinases | Cardiovascular diseases |
| | (artherosclerosis), |
| | Sclerosis, Arthritis |
| Proteosome | Parkinson disease |

In order to reduce toxic effects and increase drug specificity, approaches based on the prodrug concept, in which an inactive compound is administered to the patient and then later transformed to the pharmacologically active form of such drug through enzymatic activity have also been explored. Unfortunately, the targeting of enzymes to release a drug (usually a small molecule) in its native, active form is indeed a difficult task requiring great skill in pharmacology, biology, and synthetic organic chemistry. There is, however, a good exception in which the prodrug concept has led to important developments in the diagnosis and treatment of certain cancers, as well as in novel cosmetic applications including hair removal, the treatment of acne, or skin rejuvenation. This approach involves the targeting of abnormal enzymatic activity in the heme biosynthetic pathway of neoplasia with the use of aminolevulinic acid derivatives to accumulate endogenous photoactive porphyrins in cells and tissue. These photoactive porphyrins are then used for fluorescence diagnosis or therapeutic purposes. (For reviews see Fukuda et al., 2005; Lopez et al., 2004). However, one major drawback of aminolevulinic acid (and derivatives) is its inherent systemic toxicity, which has limited its use to mainly applications requiring its local administration.

Similarly, enzymatic targeting of neoplasia for fluorescence based diagnosis has recently met important advancements. Particularly, Weissleder and coworkers have developed “quenched” polylysine-PEG conjugates carrying near-infrared probes for systemic administration (Funovics et al., 2003; Zhou et al., 2003; Mahmood and Weissleder, 2003; Pham et al., 2004; WO 200308; WO 2002056670; WO 2002000265; WO 9958161). These probes are loaded with various amounts of Cy5.5 fluorophores and are virtually non-fluorescent due to autoquenching by energy transfer between the fluorophores. Nevertheless, the probes become fluorescent only in the presence of enzymes such as trypsin, cathepsins, and matrix metalloproteinases which are present in greater abundance in certain cancers. Unfortunately, these agents are only limited to photodetection and fail to produce any desired photochemotherapeutic outcome (in contrast, see the below examples).

In addition, other polymer quenched probes have been reported for the imaging of protease activity (see McIntyre et al., 2004; Bigelow et al., 2004).

Certain compounds comprising the polymer polylysine have been used for imaging. These compounds are limited to diagnostic applications only and fail to produce any desired photochemotherapeutic outcome (in contrast, see the below examples) (Funovics et al., 2003, Zhouet al., 2003; Mahmood, and Weissleder, 2003; Pham et al., 2004; WO 2003082988; WO 2002056670; WO 2002000265; WO 9958161; McIntyre et al., 2004).

### SUMMARY OF THE INVENTION

The present invention is based, at least in part, on the surprising observation that photosensitizing molecules, covalently attached to a polymer carrier with or without [symbol] and/or fluorescent and/or photosensitizing moieties, exhibit little or no phototoxic activity in the absence of specific target enzymes. In contrast, their phototoxicity shows a remarkable increase upon exposure to the target enzyme(s).

An aspect of the present invention relates to a pharmacologically acceptable photosensitizer conjugate comprising one or more photosensitizer moiety conjugated to a biocompatible polymer backbone, wherein the conjugate is enzyme-activatable to increase the activity of the photosensitizer. The polymer backbone may be enzyme degradable by, for example, a peptidase, a glycolytic enzyme, an esterase, a trypsin, a cathepsin, lipoprotein lipase, lecithin:cholesterol acyltransferase, 26-hydroxylase, an enzyme that regulates disorders in metabolism of porphyrins and heme, lysyl hydroxylase, collagenase, lactase, trehalase, prostate specific antigen (PSA), a matrix metalloproteinase, a CMV protease, or a proteosome. In certain embodiments, the polymer backbone is degradable by cathepsin D, cathepsin B or cathepsin H. The polymer may be a dimer, trimer, an oligomer, a copolymer, a block copolymer, or a crosslinked polymer. In certain embodiments, the polymer may comprise an oligonucleotide, polypeptide, a polysaccharide, a polyamide, a polylactide, a polyacrylamide, a polystyrene, a polyurethane, a polycarbonate or a polyester polylysine, poly-L-lysine, poly-D-lysine, polyarginine, polyornithine, polyglutamic acid, a peptide comprising L and/or D amino acids, polyvinyl alcohol, polyacrylic acid, polymethacrylate, polyacrylamide, polyalkylcyanoacrylate, polyhydroxyacrylate, polysuccinimide, polysuccinic anhydride, poly(hydroxyethyl methacrylate) (HEMA), chitosan, polyhydroxybutanoates, polyglycolic acid, copolymers of polylactides and polyglycolic acids, or polyvinyl alcohol.

In certain embodiments, an enzyme-cleavable linker is conjugated to the polymer backbone. The photosensitizer moiety or a quencher may be conjugated to the enzyme-cleavable linker. In certain embodiments, the enzyme-cleavable linker is a cathepsin D cleavable linker or an Epsilon N-amide bond. The enzyme-cleavable linker may be an amino acid sequence; for example, the amino acid sequence may comprise Gly-Thr-Phe-Arg-Ser-Ala-Gly (SEQ ID NO: 1).

In certain embodiments, the photosensitizer moiety is selected from the group consisting of chlorines, chlorophylls, coumarines, cyanines, fullerenes, metallophthalocyanines, metalloporphyrins, methylenporphyrins, naphthalimides, naphthalocyanines, nile blue, perylenequinones, phenols, pheophorbides, pheophyrins, phthalocyanines, porphycenes, porphyrins, psoralens, purpurins, quinines, retinols, rhodamines, throphenes, verdins, xanthenes, and dimers and oligomers thereof. The photosensitizer moiety may be hematoporphyrin derivative (HPD), photofrin II (PII), tetra(m-hydroxyphenyl)chlorin (mTHPC), benzoporphyrin derivative mono acid ring (BPD-MA), zinc-phthalocyanin (ZnPC), protoporphyrin IX, chlorin e6, AlS4Pc, a texaphyrin, hypericin, or pheophorbide a.

In certain embodiments, photosensitizer moieties are covalently attached to between from about 0.1% to about 80 %, or from about 3% to about 50%, of the available functionalities of the polymer. The photosensitizer moieties may be covalently attached to between of the available functionalities of the polymer.

The conjugate may further comprise one or more quencher moieties conjugated to the polymer backbone. The quencher moiety may be in sufficient proximity to the photosensitizer to reduce the activity of the photosensitizer. The quencher moiety may comprise a non-fluorescing dye, DABCYL; DANSYL, QSY-7, a black hole quencher, a fluorophore, a nano-scaled semiconductor, a quantum dot, a nanotube, a fluorophore, or a gold nanoparticle. The photosensitizers may participate in energy transfer with the quencher.

In certain embodiments, the conjugate further comprises one or more biocompatibilizing units. The biocompatibilizing unit may be polyethyleneglycol (PEG), methoxypolyethyleneglycol (MPEG), polyethyleneglycol-diacid, PEG monoamine, MPEG monoamine, MPEG hydrazine, MPEG imidazole, methoxypropyleneglycol, a copolymer of polypropyleneglycol or methoxypropyleneglycol, dextran, polylactic-polyglycolic acid, 2-(N,N,N-Trimethylammonium)ethanoic acid, 1-methyl nicotinamide, 1-methyl nicotinamide, or monosuccinamide.

In certain embodiments, the conjugate further comprises one or more protecting units that reduces the rate of enzyme-activatable release of the photosensitizer. The protecting unit may comprise an amide, an imide, an imine, an ester, a thioester, a carbazone, a hydrazone, an oxime, an acetal, a ketal derivative of N-methylnicotinic acid, N-methylquinoline-X-carboxylic acid (where X=2, 3, 4, 5, 6, or 7), a substituted N-methylbenzoquinoline, a substituted N-methylacridine, a substituted N-methyl isoquinoline, a substituted N-methylphenanthredine or an N-alkylated derivative thereof, a substituted substituted pyridine, a benzopyridine, a dibenzopyridine, a dicarboxylic acid, oxalic acid, maleic acid, succinic acid, glutaric acid, adipic acid, a polycarboxylic acid, citric acid, an amino acid, a peptide, an ammo acid or pepticle in which the amine functions are quaternized by methyl or other alkyl group, a sulfoacid, sulfoacetic acid, ascorbic acid-2-sulfate, an O-sulfonated amino acid, O-sulfo-serine, O-sulfo-tyrosine, O-sulfo-threonine, an O-sulfonated saccaride, a polysaccaride, a phosphorylated acid or amino acid, phosphogliceric acid, O-phospho-serine, O-phospho-threonine, O-phospho-tyrosine, ascorbic acid-2-phosphate, an O-phosphorylated saccaride or polysaccaride, glyceraldehyde-3-phosphate, glucose-6-phosphate, erythrose-4-phosphate, ribose-5-phosphate, pyridoxal-5-phosphate, or glusosamine-6-sulfate.

In certain embodiments, the conjugate further comprises a targeting moiety. The targeting moiety may comprise folic acid, a steroid such as cholesterol or a cholesterol ester, a cell adhesion molecule, a targeting peptide such as RGD, a saccharide, a polysaccharide, an oligonucleotide, an antibody, an antibody fragment or single chain antibody. The molecular weight of the conjugate may be between 1 kDa to 100,000 kDa

In certain embodiments, the conjugate is comprised in a pharmaceutical composition. The pharmaceutical composition may be formulated for parenteral administration to a human.

Another aspect of the present invention relates to a method of photochemotherapy comprising administering the conjugate of the present invention to a subject (e.g., a human patient) in an effective amount. The method may comprise treating a disease, such as acne, a cell proliferative disease, a bacterial disease, a viral disease, a fungal infection, age-related macular degeneration, diabetic retinopathy, an arthritic disease, an inflammatory disease such as rheumatoid arthritis, neovascularization, cancer, psoriasis, skin cancer, or actinic keratosis.

In certain embodiments, the method is performed for a cosmetic purpose, such as hair removal or skin rejuvenation. The administration may be topical or systemic. The method may further comprise irradiation of part or all of the subject. The irradiation may be carried out at a wavelength that is an absorption wavelength of the photosensitizer, for example, between from about 350 to about 800 nm. The wavelength may be in the blue region, the red or near-infrared region, white light. The irradiation may be carried out by a light source equipped with a filter. The irradiation may be performed with a laser. The irradiation may be performed within a time interval of 4 minutes to 168 hours, 4 minutes to 72 hours, or 15 minutes to 48 hours after administration of the conjugate. The total fluence of light used for irradiation may be between 2 J/cm² and 500 J/cm².

It is an object of the present invention to overcome drawbacks and limitations of conventional and/or conjugated photosensitizing agents discussed above.

Another object of this invention is to prepare photosensitizer-polymer conjugates that exhibit phototoxic effects only upon exposure to a specific enzyme, but none or only limited phototoxicity when in its native form.

A further object of the invention is to offer a general methodology to directly use identified overexpression of enzymes for therapeutic purposes.

One object of this invention is to use methods of enzyme-activatable photosensitizer-polymer conjugates, or compositions or formulations of it for photochemotherapeutic purposes.

One additional object of this invention is to use said enzyme-activatable photosensitizer-polymer conjugates wherein irradiation is performed quickly and without considerable delay.

A further object of this invention is the selective destruction of target cells and tissues via photochemotherapeutic action using said enzyme-activatable photosensitizer-polymer conjugates in vivo and in vitro.

Another object of the present invention is to use said enzyme-activatable photosensitizer-polymer conjugates and methods to enable the treatment of cells or tissues expressing in abundances a target enzyme without the use of expensive equipment.

Another aspect of this invention is the use of said enzyme-activatable photosensitizer-polymer conjugates that are fluorescently or otherwise labeled in order to determine their presence in the target tissue.

A further object of the invention is the use of pharmaceutically acceptable formulations and compositions of enzyme-activated photosensitizer-polymer conjugates that enable systemic or topical administration of said conjugates.

Another object of this invention is the use of enzyme-activated photosensitizer-polymer conjugates that are coupled to moieties that facilitate the cellular uptake of said conjugates.

Another object of this invention is the use of enzyrne-activatable photosensitizer-polymer conjugates that are coupled to moieties that improve solubility, and/or biocompatibility, and/or stability of said conjugates.

A further object of this invention includes kits that can be used to make enzyme-activatable photosensitizer-polymer conjugates for the targeting of specific enzymes.

Furthermore, an object of this invention is the use of said enzyme-activatable photosensitizer-polymer conjugates in combination with penetration enhancers.

Another object of the invention is the use of said conjugates in combination with therapeutic or phototherapeutic agents.

A further object of the invention includes said conjugates, in which the backbone is a natural or synthetic polymer with or without further modifications to affect the stability, and/or physicochemical properties of the polymer.

A further object of the invention includes said conjugates, in which the backbone is a natural or synthetic polymer with or without further modifications to introduce or modified existing side-chain functionalities.

The terms “inhibiting,” “reducing,” or “prevention,” or any variation of these terms, when used in the claims and/or the specification includes any measurable decrease or complete inhibition to achieve a desired result.

The term “effective,” as that term is used in the specification and/or claims, means adequate to accomplish a desired, expected, or intended result.

The use of the word “a” or “an” when used in conjunction with the term “comprising” in the claims and/or the specification may mean “one,” but it is also consistent with the meaning of “one or more,” “at least one,” and “one or more than one.”

It is contemplated that any embodiment discussed in this specification can be implemented with respect to any method or composition of the invention, and vice versa. Furthermore, compositions of the invention can be used to achieve methods of the invention.

Throughout this application, the term “about” is used to indicate that a value includes the inherent variation of error for the device, the method being employed to determine the value, or the variation that exists among the study subjects.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF ILLUSTRATIVE EMBODIMENTS

The present invention benefits from recent progress made in the field of fluorescence diagnostics. It is based on our own surprising observation that the phototoxicity of photosynthesizers can be greatly reduced by loading them in relative close proximity on a polymer carrier. In this configuration, the photosensitizer moieties undergo efficient energy transfer and autoquench their triplet excited state, which renders them inactive toward the production of reactive oxygen species (ROS) or other active radical and non-radical molecules. Another possibility is that the presence of a molecular group hinders the collisional energy transfer between the photosensitizer and a third molecule, such as molecular oxygen. The present invention relates to the field of photochemotherapy, polymer chemistry, peptide chemistry, cell biology, biology, organic chemistry and physical chemistry. Methods, kits and compositions described in the present invention can be used for the selective destruction of cells and tissular structures expressing specific enzymes. They may be used [symbol] , cosmetically, in vitro and in vivo, as well as in bacteriology, virology, food technology and agriculture.

The family of enzyme-activatable photosensitizing conjugates in this invention may comprise six main components, which do not all have to be present in the conjugate to obtain the desired results; they are the following: 1) polymer carrier, 2) photosensitizers, 3) quenchers, 4) targeting moieties, 5) protecting units, and 6) biocompatibilizing units.

The two indispensable components required to construct these enzyme-activatable conjugates are the polymer backbone and the photosensitizer moieties. For instance, the use of polyamide (polylysine or polyglutamic acid, etc) or polyester backbones for example, can be used directly for the targeting of either peptidases or esterases. Thus, the targeting is achieved by enzyme specific backbone degradation of the conjugate, which liberates fragments containing fewer photosensitizer units which are activated towards production of ROS and other reactive molecules. Similarly, oligosaccharide and oligonucleotide backbones can be used in a similar fashion.

Another component of these conjugates is an enzyme targeting linker. These molecules provide a stable covalent bond between the polymer and the photosensitizer, but are easily cleaved by specific enzymes. They provide a somewhat more advantageous conjugate architecture, in which the linkers rather than the polymer backbone are degraded by target enzymes, and thus, they permit higher photosensitizer loadings on the polymer as well as finer tuning of an enzyme-targeting sequence.

As mentioned above, the targeting of enzymes is accomplished in either of two ways. The first possibility is to use, for instance, poly-L-lysine conjugates in which the backbone (polylysine) can be degraded by certain enzymes such as trypsin, or cathepsins (they cleave by recognizing KK). The other possibility to target enzyme activity involves the use of a stable or partially stable polymer backbone with enzyme-cleavable linkers between the polymer and the photosensitizers. In this case, activation of the conjugate is accomplished by the use of enzyme-specific peptide sequences, saccharides, polysaccharides, polyesters, oligonucleotides, or any other synthetic or natural molecule that is a substrate for a target enzyme.

Furthermore, it is possible to install “quencher” units, which comprise additional fluorescent or non-fluorescent photosensitizers, fluorescent or non-fluorescent quenchers, [symbol] quenchers, quantum dots, gold nanoparticles etc. to obtain an “inactive chromophore combination”. This “inactive chromophore combination” comprises two or more groups of photosensitizers and/or chromophores in which the units participate in energy transfer. Typically, one of the groups acts as a photosensitizer moiety while the other acts as an excited energy modifying moiety. Thus, this chromophore arrangement provides more efficient quenching of the conjugate. Finally, the use of “inactive chromophore combinations” allows for the targeting of more than one enzyme.

Additional functionalities installed on the conjugate include targeting moieties, which include but are not limited to folic acid, cholesterol esters, cell adhesion molecules (RGD peptides, etc.), saccharides, polysaccharides, oligonucleotides, antibodies, etc. The targeting moieties are there to improve the selectivity of the conjugates towards a specific tissue or pathology. The attachment between the polymer and the targeting moiety might be a covalent or a non-covalent bond.

Furthermore, additional “protecting” functionalities that alter the pharmacokinetic properties and protect the polymeric backbone against unwanted enzymatic attack may be installed on the conjugate. For example, biocompatible, small organic substituents may increase the water-solubility of the polymer and may serve as biocompatibilizing unites. These substituents typically carry a permanent charge under physiological conditions. Small organic substituents are well known to persons skilled in the art.

Finally, biocompatibilizing units, such as but not limited to mPEG, or PEG chains with molecular weight ranging from 1 kDa to 20 kDa, but more preferably between 2 kDa to 5 kDa, are used to impart good water solubility to the conjugate, minimize non-specific ionic interactions with tissue, and suppress unwanted inununological responses. Besides PEG-derived polymers and copolymers, it is also possible to use dextrans or polysaccharides to accomplish the same goal.

The invention also includes pharmaceutical compositions of said photosensitizer polymer conjugates together with at least one pharmaceutical carrier or exipient. Such pharmaceutical composition can be made for either topical, or systemic application (e.g., oral, inhalational, intravenous, or intraperitoneal administration).

Furthermore, the invention includes kits of said enzyme-activated polymer conjugates for photochemotherapeutic purposes in vivo and in vitro comprising:

a) a first container containing said photosensitizer polymer conjugates or a solution of said photosensitizer polymer conjugates;

b) a second container with at least one solubilizing pharmaceutically acceptable carrier.

Furthermore, the invention comprises methods, using at least one enzyme-activatable photosensitizer conjugate according to this invention as an active compound for therapeutic purposes. Methods according to this invention may be performed in vivo and in vitro. Our most preferred methods are performed in vivo. However, under certain conditions including sterilization, methods according to this invention may be performed in vitro. By sterilization, the inventors mean blood purging, destruction of viruses and bacteria in food industry, medicine, and agriculture.

A method to destroy or impair cells expressing the target enzyme typically comprises the following steps:

a) topical or systemic administration of a therapeutically effective amount of said enzyrne-activatable photosensitizer conjugate in a pharmaceutically acceptable composition according to this invention

b) permitting sufficient time to elapse, allowing the uptake of an effective amount of photosensitizer conjugate according to this invention in the target

c) irradiation of a target area of the subject with light having a wavelength corresponding at least in part to the absorption bands of the enzymatically cleaved photosensitizers.

### I. Definitions

As used herein, “polymer” means a material made of two or more covalently linked monomer units in a linear or nonlinear fashion. This definition includes dimers, trimers, and higher oligomers, as well as copolymers, block copolymers, and crosslinked polymers. Examples of some useful polymers that may be used with the present invention include polylysine, poly-L-lysine, poly-D-lysine, polyarginine, polyornitine, polyglutamic acid, peptides comprised of L and/or D amino acids, as well as those comprised of unnatural amino acids, polyvinyl alcohol, polyacrylic acid, polymethacrylate, polyacrylamide, [symbol] , polyhydroxyacrylate, polysuccinimide, polysuccinic anhydride, poly(hydroxyethyl methacrylate) (HEMA), polysaccharides, oligonucleotides, and chitosan. Also included are polymers that have been modified with additional functionalities in the side chain or the backbone to impart desired physicochemical properties and/or sites for covalent attachment to other molecules such as polystyrene, polystyrene-maleic anhydride, polyesters, polycarbonates, polylactides, polyurethanes, polyethelene, polydivinylbenzene, chitosan-cysteine, chitosan-thioglycolic acid, chitosan-4-thiobutylamidine, polycarbophilcysteamine, and polycarbophil-cysteine. Polymers of the present invention exclude dendrimers (also called a “cascade molecule”, a polymer in which the atoms are arranged in many branches and subbranches along a central backbone of carbon atoms). The examples given here are only illustrative and by no means limit or exclude this patent from the use of other polymers.

“Enzyme-cleavable linker” or “enzyme clevable linker”, as used herein, refers to a monomer or polymer unit which serves as a covalent bond between the polymer and a desired moiety, such as a photosensitizer, a fluorescent photosensitizer, a non-fluorescent photosensitizer, a chromophore, a fluorophore, a quencher, a blackhole quencher, a gold nanoparticle, a quantum dot, or a iron oxide nanoparticle. The examples given here are only illustrative and by no means limit or exclude this patent fiom the use of other moieties. The enzyme-cleavable linker might be a natural or unnatural amino acid, a peptide made of L and/or D amino acids, a peptide made of unnatural amino acids, a polysaccharide, an oligonucleotide, an oligonucleotide with modified nucleobases and/or modified backbone, or a natural or synthetic molecule which serves as an enzymatic substrate. The examples given here are only illustrative and by no means limit or exclude this patent from the use of other linkers.

As used herein, “functional group” refers to an organic moiety with the potential to either undergo a useful transformation, such as to make a covalent bond, or with the potential to serve a useful purpose, such as impart desired solubility, suppress enzymatic attack, suppress immunological responses, etc. Examples of potentially useful functional groups include but are not limited to olefins, acetylenes, alcohols, phenols, ethers, oxides, halides, aldehydes, ketones, carboxylic acids, esters, arnides, cyanates, isocyanates, thiocyanates, isothiocyanates, amines, hydrazines, hydrazones, hydrazides, diazo, diazonium, nitro, nitrol, mercaptanes, sulfides, disulfides, sulfoxides, sulfones, sulfonic acids, sulfinic acids, acetals, ketals, anhydrides, sulfates, sulfenic acids, amidines, imides, nitrones, hydroxylamines, oximes, nyaroxamic acids, thiohydroxamic acids, allenes, ortho esters, sulfites, enamines, amines, ureas, pseudo ureas, semicarbazides, carbodiimides, imines, azides, azo compounds, azoxy compounds, and nitroso compounds.

As used herein, “nucleic acid” means DNA, RNA, singled-stranded, double-stranded, or more highly aggregated hybridization motifs, and any chemical modification thereof. Modifications include, but are not limited to, those providing chemical groups that incorporate additional charge, polarizability, hydrogen boding, electrostatic interaction, and fluxionality to the nucleic acid ligand bases or to the nucleic acid ligand as a whole. The nucleic acid may have modified intemucleotide linkages to alter, for example, hybridization strength and resistance to specific and non-specific degradation. Modified linkages are well-known in the art and include, but are not limited to, methylphosphonates, phosphothioates, phosphodithionates, phospoamidites, and phosphodiester linkages. Alternatively, dephospho-linkages, also well-known in the art, can be introduced as bridges. These include, but are not limited to, siloxane, carbonate, carboxymethylester, acetamide, carbamate, and thioether bridges.

The term “amino acid” as referred herein, means a naturally occurring with either L or D configuration or synthetic amino acid as understood by persons skilled in the art. It also includes amino acid with additional substituents in the alpha position or side chains. It also includes amino acids with unnatural side chains. It also includes amino acids in which additional methylene units have been introduced into the backbone, such as beta, gamma, delta, etc. amino acids. It also includes cyclic amino acids in which additional methylene units have been introduced on the backbone or side chains. All other amino acid mimics included in this definition will be obvious to one skilled in the art.

As used herein, “peptides”, refer to a polymer of amino acids. They also include peptidomimetics, in which either natural or synthetic amino acids are linked by either amide bonds or non-amide bonds (such as peptoids, etc).

“Proteins” as used herein, refers to a linear or non-linear polymer of peptides. Proteins include, but are not limited to, enzymes, antibodies, hormones, carriers, etc. without limitation.

As used herein, “biocompatibilizing units” refers to any natural or synthetic moiety that is introduced to one of the different components of the enzyme-activable photosensitizer in order to alter its pharmacokinetic profile, modify its biodistribution or clearance, and to protect the polymeric backbone from unwanted degradation. Examples for such entities are well known in the art and include but are not limited to polyethylene glycol, polyethylene glycol copolymers, dextrans, cyclodextran, saccarides, polysaccarides etc

“Protecting. units”, as used herein, are small chemical entities of natural or synthetic origin, that serve to shield the polymeric backbone from enzymatic degradation by masking key enzymatic recognition sites of the substrate. These protecting units include but are not limited to amide, imide, imine, ester, thioester, carbazone, hydrazones, oxime, acetal, and ketal derivatives of N-methylnicotinic acid, N-methylquinoline-X-earboxylic acid (where X=either 2, 3, 4, 5, 6, or 7), substituted N-methylbenzoquinolines, substituted N-methylacridine, substituted N-methyl isoquinoline, substituted N-methylphenanthredines or any other N-alkylated derivative thereof. Protecting units also include substituted N-alkylated pyridine containing systems, such as substituted pyridines, benzopyridines, dibenzopyridines, etc. These substituents include but are not limited to carboxylic acid and esters, aldehydes, ketones, amines, alcohols, etc. These protecting units also include monovalent derivatization with dicarboxylic acids, including oxalic, maleic, succinic, glutaric, adipic acid, etc., or polycarboxylic acids, including citric acid etc., or natural or unnatural amino acids or peptides, in which the amine functions may or may not be quaternized by methyl or any other alkyl group. Alkylation of amines can also be used to quatemize polymeric amine functions. Other protecting functionalizations include derivatization with sulfoacids (e.g., sulfoacetic acid, ascorbic acid-2-sulfate, etc.), 0-sulfonated amino acids (e.g., O-sulfo-serine, O-sulfo-tyrosine), O-sulfo-threonine, O-sulfonated saccarides, polysaccarides or peptides. Similarly, derivatization may be performed using phosphorylated acids or amino acids (e.g., phosphogliceric acid, O-phospho-serine, O-phospho-threonine, O-phospho-tyrosine, ascorbic acid-2-phosphate “vitamin C phosphate”), O-phosphorylated saccarides or polysaccarides (e.g., glyceraldehyde-3-phosphate, glucose-6-phosphate, erythrose-4-phosphate, ribose-5-phosphate, pyridoxal-5-phosphate, glusosamine-6-sulfate, etc.).

As used herein, “targeting moiety” refer to any natural or synthetic molecule with the potential to bind in a covalent or a non-covalent fashion to a receptor, antibody, antigen, protein, cell membrane, or tissue of interest. Targeting moieties include peptides, peptides with L and/or D configured amino acids, peptides with unnatural amino acids, cell adhesion molecules (RGD peptides and peptide mimetics, etc), steroids, modified steroids, saccharides, [symbol] , folic acid, cholesterol, cholesterol esters, and antibodies. The examples given here are only illustrative and by no means limit or exclude this patent from the use of other targeting moieties.

As used herein, “target” refers to any molecule; enzyme, receptor, cell membrane, protein, antibody, antigen, tissue, or pH of interest. A specific target is chosen to impart greater selectivity to the conjugate by improving its affinity towards pathological regions. For instance, neoplastic cells can be selectively targeted by exploiting overexpression of cell adhesion receptors (RGD, etc), folic acid receptors, LDL receptors, insulin receptos and/or glucose receptors; in addition, neoplastic cells are known to express cancer specific antigens. A target can also be, for example, an enzyme (metallomatrix proteases, cathepsin, etc), nucleic acid, peptide, protein, polysaccharide, carbohydrate, glycoprotein, hormone, receptor, antibody, virus, substrate, metabolite, cytokine, inhibitor, dye, growth factor, nucleic acid sequence, pH value, and so on.

As used herein, “photosensitizer” refers to molecules, which upon irradiation with light having a wavelength corresponding at least in part to the absorption bands of said “photosensitizer” interact through energy transfer with another molecule to produce radicals, and/or singlet oxygen, and/or ROS. Photosensitizing molecules are well-known in the art and include lead compounds, including but not limited to, chloiines, chlorophylls, coumarines, cyanines, fullerenes, metallophthalocyanines, metalloporphyrins, methylenporphyrins, naphthalimides, naphthalocyanines, nile blue, perylenequinones, phenols, pheophorbides, pheophyrins, phthalocyanines, porphycenes, porphyrins, psoralens, purpurins, quinines, retinols, rhodamines, thiophenes, verdins, xanthenes, and dimers and oligomers thereof. The term “photosensitizer” also includes photosensitizer derivatives; for example, the positions in a photosensitizer may be functionalized by an alkyl, functional group, peptide, protein, or nucleic acid or a combination thereof.

As used herein, “quenching”, refers to a process by which the energy of an excited state of a molecule or at least part of such energy, is altered by a modifying group, such as a quencher. If the excited energy of the modifying group corresponds to a quenching group, then one of the excited triplet states or singlet states of the photosensitizer is depopulated. If the excited energy of the modifying group corresponds to a large molecule, by which the inventors mean compounds of several hundred Daltons, the energy transfer between the photosensitizer and a third molecule or atom is hindered. It is understood by persons skilled in [symbol] compound to obtain the desired quenching fluorescent complement. In addition [symbol] , naturally occurring fluorescent proteins and engineered analogues of such proteins are useful in the present invention. Such proteins include, for example, green fluorescent proteins of cnidarians (Ward et al., 1982; Levine et al., 1982), yellow fluorescent protein from Vibriofischeri strain (Baldwin et al., 1990), Peridinin-chlorophyll from the dinoflagellate Symbiodinium sp. (Morris et al., 1994), phycobiliproteins from marine cyanobacteria, such as Synechococcus, e.g., phycoerythrin and phycocyanin (Wilbanks et al., 1993), and the like.

c) Photosensitizers (definition see above)

d) Nano-scaled semiconductors, such as quantum dots, nanotubes, and other quantum-well structures.

“Pharmaceutical Composition” as used herein, means a formulation of compounds or complexes according to this invention in conventional manner with one or more physiologically acceptable carrier or excipient, according to techniques well-known in the art. They may be applied systemically, orally or topically. Topical compositions include, but are not limited to, gels, creams, ointments, sprays, lotions, salves, sticks, soaps, powders, pessaries, aerosols, and other conventional pharmaceutical forms in the art. Ointments and creams may, for example, be formulated with an aqueous or oily base with the addition of suitable thickening and/or gelling agents. Lotions may be formulated with an aqueous or oily base and will, in general, also contain one or more emulsifying, dispersing, suspending, or thickening agent. Powders may be formed with the aid of any appropriate powder base. Drops may be formed with an aqueous or non-aqueous base containing, sometimes, one or more emulsifying, dispersing, or suspending agents. Alternatively, the compositions may be provided in an adapted form for oral or parenteral administration, including intradermal, subcutaneous, intraperitoneal, or intravenous injection. Thus alternative pharmaceutically acceptable formulations include plain or coated tablets, capsules, suspensions and solutions containing compounds according to this invention, optionally together with one or more inert conventional carriers and/or diluents, including, but not limited to, corn starch, lactose, sucrose, microcrystalline cellulose, magnesium stearate, polyvinyl-pyrrolidone, citric acid, tartaric acid, water, water/ethanol, water/glycerole, water/sorbitol, water/polyethylenglycol, propylengycol, water/propyleneglycol/ethanol, water/polyethylenegycol/ethanol, stearylglycol, carboxymethylcellulose, phosphate buffer solution, or fatty substances such as [symbol] or suitable mixtures thereof. Altematively, the compounds according to the invention may be provided in liposomal formulations. Pharmaceutically acceptable liposomal formulations are well-known to persons skilled in the art and include, but are not limited to, phosphatidyl cholines, such as dimyristoyl phosphatidyl choline (DMPC), phosphatidyl choline (PC), dipalmitoyl phosphatidyl choline (DPPC), and distearoyl phosphatidyl choline (DSP), and phosphatidyl glycerols, including dimyristoyl phosphatidyl glycerol (DMPG) and egg phosphatidyl glycerol (EPG). Such liposomes may optionally include other phospholipids, e.g. phosphatidyl ethanolamine, phosphatic acid, phosphatidyl serine, phosphatidyl inositol, abd disaccarides or poly saccarides, including lactose, trehalose, mattose, maltotriose, palatinose, lactulose, or sucrose in a ratio of about 10-20 to 0.5-6, respectively.

The phrases “pharmaceutical” or “pharmacologically acceptable” refers to molecular entities and compositions that do not produce an adverse, allergic or other untoward reaction when administered to an animal, such as, for example, a human, as appropriate. Of course, what is pharmaceutically acceptable may vary based on the route of administration; for example, a broader range of polymers may be used with the present invention for topical administration, as compared to certain other routes of administration (e.g., parenteral). The preparation of a pharmaceutical composition that contains at least one photosensitizer conjugate of the present invention or additional active ingredient will be known to those of skill in the art in light of the present disclosure, as exemplified by Remington's Pharmaceutical Sciences, 18^{th} Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18^{th} Ed. Mack Printing Company, 1990, pp. 1289-1329, incorporated herein by reference). Except insofar as any conventional carrier is incompatible with the active ingredient, its use in the pharmaceutical compositions is contemplated.

### II. Enzyme-Activatible Photosensitizers

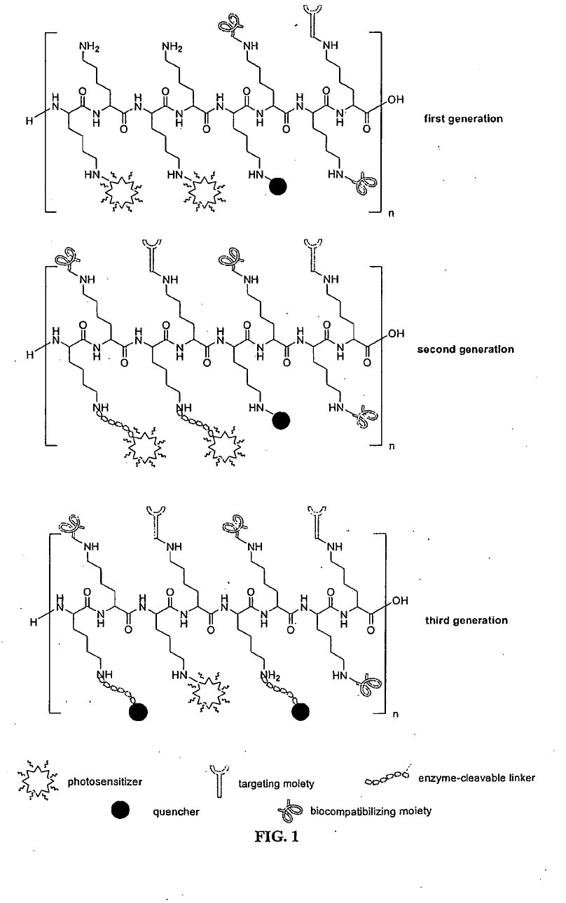

An illustration of first generation (1), second generation (2), and (3) third generation enzyme-activatable photosensitizer-polymer conjugates is provided in FIG. 1 (using a polylysine backbone as one possible example). All of said conjugates have a basic common construct, namely a polymeric backbone with suitable functional groups to which photosensitizer units are attached.

Enzymes that may be targeted with an enzyme-activatable photosensitizer-polymer conjugates include, for example, lipoprotein lipase, lecithin:cholesterol acyltransferase, 26-hydroxylase (cholesterol), enzymes that regulate disorders in metabolism of porphyrins and heme, lysyl hydroxylase, collagenase, lactase, trehalase, cathepsin D, cathepsin B, cathepsin H, prostate specific antigen (PSA), matrix metalloproteinases, CMV protease, and proteosomes. It is further anticipated that, for example, virtually any enzyme listed in Table I may be used with the present invention.

First generation conjugates (1) have a targeting system based on enzymatic degradation of its polymeric backbone. Thus, this requires not only that the polymeric backbone is an enzymatic substrate, such as polyamides (poly-L-lysine, polyarginine, peptides, proteins, etc.), polyesters (polylactic acid, polylactides, polyhydroxybutanoates, etc.), polysaccharides, etc. but also that introduced modifications to the polymer by either introducing functional groups on the backbone or simply by modifying preexisting functional groups does not completely impede its enzymatic degradation. Thus, first generation conjugates do not necessarily require specialized enzyme targeting linkers and the tethering of the photosensitizers is accomplished with any “stable” covalent bond used by those skilled in the art. First generation conjugates could also have three additional features. The first feature is the use of “quenchers” (see definition) that will improve on the autoquenching of the conjugate due to more efficient energy transfer between the photosensitizer and the quencher units. The second feature is the use of targeting moieties such as cell adhesion molecules, folic acid, glucose, cholesterol, antibodies, etc. to increase the selectivity of the conjugate towards the target cells or tissues where the target pathology is present. Finally, the third feature includes the use of biocompatibilizing and protecting molecules such as mPEG, PEG, [symbol] , polysaccharides, N, methylated amino acids, N-methylated nicotinic acid, succinic acid, etc. to impart better solubility to the conjugate, suppress unwanted immuno responses, minimize non-specific ionic interactions with tissue, to increase circulation times, and to reduce non-specific enzymatic degradation. It should be noted that these components can be used in a variety of combinations which will be obvious to those skilled in the art and manipulated to fit a specific application for which it is intended.

Tethering of units to the polymer backbone is accomplished through covalent bonds which are preferably made under mild reaction conditions. Reactive groups and classes of reactions useful in practicing the present invention are generally those that are well known in the art of bioconjugate chemistry. Currently favored classes of reactions available are those which proceed under relatively mild conditions. These include, but are not limited to nucleophilic substitutions (e.g., reactions of amines, thiols and alcohols with acyl halides, active esters, and carbon-halide bonds), electrophilic substitutions (e.g., enamine reactions) and additions to carbon-carbon and carbonheteroatom multiple bonds (e.g., Michael reaction, Diels-Alder addition).

Useful reactive functional groups include, for example:

a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters;

b) hydroxyl groups, which can be converted to esters, ethers, aldehydes, etc.

c) haloalkyl groups, wherein the halide can be later displaced with a nucleophilic group such as, for example, an amine, a carboxylate anion, thiol anion, carbanion, or an alkoxide ion, thereby resulting in the covalent attachment of a new group at the site of the halogen atom;

d) dienophile groups, which are capable of participating in Diels-Alder reactions such as, for example, maleimido groups;

e) aldehyde or ketone groups, such that subsequent derivatization is possible via formation of carbonyl derivatives such as, for example, imines, hydrazones, semicarbazones or oximes, or via such mechanisms as Grignard addition or [symbol] addition; sulfonyl halide groups for subsequent reaction with amines, for example, to form sulfonamides;

g) thiol groups, which can be, for example, converted to disulfides or reacted with acyl halides;

h) amine or sulfhydryl groups, which can be, for example, acylated, alkylated or oxidized;

i) alkenes, which can undergo, for example, cycloadditions, acylation, Michael addition, etc;

j) epoxides, which can react with, for example, amines and hydroxyl compounds; and

k) phosphoramidites and other standard functional groups useful in nucleic acid synthesis.

Second generation conjugates (2) have a targeting system based on enzymatic degradation of cleavable linkers tethering the photosensitizers to the polymer. Thus, this approach no longer requires the use of enzymatically degradable polymeric backbones. However, it is preferred that the polymer backbone is sufficiently stable to enzymatic attack but biodegradable. It is possible to use polyamides (poly-D-lysine, poly-L-lysine, polylysine, polyarginine, polyornitine, peptides composed of L and/or D configured amino acids and/or unnatural amino acids, proteins containing L and/or D amino acids and/or unnatural amino acids, etc.), polyesters (polylactic acid, polylactides, polyhydroxybutanoates, etc.), polyurethanes, polycarbonates, polystyrene, polyvinyl alcohol, polyacrylamides, polysaccharides, chitosan, etc. for this application. Introduced modifications to the polymer by either introducing functional groups on the polymer backbone or simply by modifying preexisting functional groups does not impede its enzymatic activation and thus loading on the polymer can be more extensive than in first generation conjugates. As it is the case with first generation conjugates, second generation conjugates could also carry any or all of the three additional features: a quencher, a targeting moiety, a protecting unit, and/or a biocompatibilizing unit. It should be noted that these components can be used in a variety of combinations which will be obvious to those skilled in the art and manipulated to fit a specific application for which it is intended.

Enzyme cleavable linkers can be any natural or synthetic molecule that is an enzymatic substrate. It is however preferred to use specific peptide sequences for this purpose which can be easily assembled on the solid-phase by the Fmoc or Boc strategy. The photosensitizer units can easily be installed on the peptide via terminal or side chain NH₂ functions (using activated esters of a photosensitizer, Michael additions, etc.), as well as OH, SH, and carboxylic functions. It is also possible to use modified or unnatural amino acids to connect the peptide to the photosensitizer which will expand the repertoire of functional groups and chemical reactions (For reviews see: Koehn and Breinbauer, 2004; Breinbauer and Koehn, 2003; Kolb et al., 2003; Veronese et al., 1999; Means and Feeney, 1998; Mattoussi et al., 2004; Hoffman and Stayton, 2004). It is also advantageous to tether the photosensitizer to the peptide on the solid-phase. This procedure offers the possibility of carrying out the coupling reaction of the photosensitizer chemoselectively on a fully or partially protected peptide, then subsequent release from the solid phase yields the enzyme-cleavable linker with the photosensitizer already installed (this synthesis will be illustrated in this publication in latter sections). Nevertheless, it is also possible to first obtain a fully deprotected peptide and cany out the coupling to the photosensitizer chemoselectively in solution (Licha et al., 2002; Rau et al., 2001; Ching-Hsuan et al., 1999). Similarly, other enzyme cleavable linkers can be employed including saccharides, polysaccharides, polyesters, and oligonucleotides to target a known over expressed enzyme which is associated with a targeted pathology (see table 1).

In the case of oligonucleotides linkers (serving as enzyme-cleavable linkers), they can be synthesized by a number of different approaches including commonly known methods of solid-phase chemistry. Conventionally, the linkers bearing a photosensitizer in one end and a spacer with the appropriate functional group at the opposite end can be synthesized on an automated DNA synthesizer (e.g. P.E. Biosystems Inc. (Foster Clif, Calif.) model 392 or 394) using standard chemistry, such as phosphoramidite chemistry (Ozaki and McLaughlin, 1992; Tang and Agrawal, 1990; Agrawal and Zameenik, 1990; Beaucage, 1993; Boal et al., 1996). When using automated DNA synthesizers, the photosensitizer and spacers are preferentially introduced during automated synthesis. Alternatively, one or more of these moieties can be introduced either before or after automated synthesis. Additional strategies for conjugation to growing or complete sequences will be apparent to those skilled in the art.

Following automated synthesis it is preferred that the reaction products will be cleaved from their support, protecting groups removed and the liker-photosensitizer be purified by methods known in the art, e.g. chromatography, extraction, gel filtration, or high pressure liquid chromatography (HPLC).

The enzyme-cleavable linker must be tethered to the conjugate chemoselectively, for this purpose a chemoselective functional group pair must be properly chosen and include but are not limited to thiols-substitution reactions (carbon-halide bonds, alkylsulphonic esters), thiols-Michael additions (acrylates, vinylsulphones, vinylketones, etc) thiols-thioligation or natural chemical ligation (requires either an N-terminal cysteine with a thioester, or 1-hydroxy-8-sulfenyl dibenzofuran moiety with a thiol, or aminoethane sulphonyl azides with thio acids, etc.), thiol-disulfide bonds, amines-substitution reactions (activated carbon-halide bonds, activated esters, and activated alkylsulphonic esters), amines-Michael additions (acrylates, vinylsulphones, vinylketones, etc), diels-alder reactions (requiring a diene and a dienophile), 1,3-dipolar additions, etc. The proper choice of chemoselective reaction will be obvious to one skilled in the art. (For reviews see: Koehn and Breinbauer, 2004; Breinbauer and Koehn, 2003; Kolb et al., 2003; Veronese and Morpurgo, 1999; Means and Feeney, 1998; Mattoussi et al., 2004; Hoffman and Stayton, 2004).

Third generation conjugates (3) have a targeting system based on enzymatic degradation of enzyme-cleavable linkers tethering “quenchers” (energy transfer modifying groups) to the polymer. Thus, in this approach phototoxixity is activated by cleaving the “quencher” moieties from the polymer rather than the photosensitizer units. This approach has two main advantages, the first one aimed to improve the physico-chemical propelties of a photosensitizer of interest and the second aimed to allow for the targeting of multiple enzymes. Nevertheless, this application requires that the loading of the photosensitizer is below the energy transfer limit for autoquenching (loading is preferably between 0.1-50% depending on the polymer backbone and loading of biocompatibilizing units). For instance, it is known that certain photosensitizers, such as pheophorbide a have limited water solubility. Thus, by permanently linking such molecules to a water soluble polymer carrier, it is assured that good water solubility will be retained during and after the enzymatic degradation. The second advantage of such conjugate architecture is that by linking the quenching units with different enzyme-cleavable linkers, allows for the targeting of multiple enzymes. It is also possible to accomplish this goal with second generation conjugates but the effect of targeting [symbol] this case will be merely additive rather than exponential. Furthermore, as it is the case with first and second generation conjugates, third generation conjugates could also carry one or both of the additional features: a targeting moiety, a protecting unit and/or a biocompatibilizing unit. It should be noted that these components can be used in a variety of combinations which will be obvious to one skilled in the art and manipulated to fit a specific application for which it is intended.

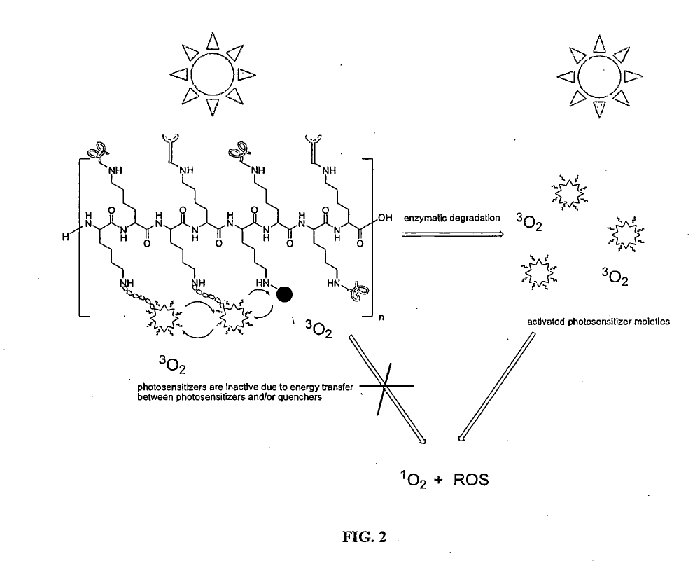

FIG. 2 depicts the principle mechanism for selective phototoxic action. In the absence of a target enzyme, the photosensitizer-conjugate remains intact in its non-phototoxic state due to effective energy transfer between photosensitizers or photosensitizers and energy modifying groups (quenchers). Hence, even upon light irradiation, the conjugate is not able to transfer, or at least only to transfer a small fraction of the energy absorbed by the photosensitizers in an excited state to a third molecule, herein represented exemplarily by molecular oxygen in its ground state. It is said that the photosensitizer-polymer conjugate is phototoxically inactive. In contrast, in the presence of a target enzyme, the conjugate undergoes degradation of either the backbone (first generation conjugates) or the cleavable linkers liberating photosensitizer fragments that are effectively further apart from each other and fully or partially activated. Hence, upon irradiation with light, a much greater ratio of the absorbed energy can then be transferred to other molecules including oxygen. In the case of oxygen, a highly reactive oxygen species in its excited singlet state (singlet oxygen) will be generated. The generation of sufficient amounts of reactive phototoxic molecules from the activated conjugate fragments may eventually lead to cell death. It is said that the photosensitizer conjugate is phototoxically active. For persons skilled in the art, it is apparent that mechanisms other than energy transfer between molecular oxygen and the phototoxically active photosensitizer conjugate may lead to cell death, e.g. the direct formation of other radicals. Furthermore, it is evident that subsequently to the formation of singlet oxygen, other reactive oxygen species may be generated and further contribute to the destruction of cells over expressing the target enzyme.

Kits according to this invention may contain one or more photosensitizer-polymer conjugates and instructions for their preparation. Optionally, kits according to this invention may include enzymes, reagents and other devices so that the user of the kit may easily use it for the preparation of photosensitizer-polyrner conjugates directed against a preselected enzyme target.

Sometimes it may be difficult to introduce polymer conjugates into the cell or to body areas where an over express target enzyme might be located. Therefore, an already mentioned important aspect of this invention is the use of effective delivery systems (targeting moieties), which allow for intracellular bioavailability of said conjugates at levels required for effective in vivo and in vitro PCT. Such molecular complex comprises a targeting moiety that is either covalently bound (see first, second, and third generation conjugates above) or non-covalently bound to the photosensitizer-conjugate according to the invention. The complex is administered in a pharmaceutically acceptable solution in an amount sufficient to perform photochemotherapy in the region of interest. The ligand binding targeting moiety (targeting moiety) includes any cell surface recognizing molecule or any molecule with a specific affinity for a cell surface component. The cell surface component can be those generally found on any cell type. Preferably, the cell surface component is specific to the cell type targeted. More preferably, the cell surface component also provides a pathway for entry into the cell, for entire conjugate. Preferably, the tethering of the targeting moiety to the conjugate does not substantially impede its ability to bind its target or its entry into the cell. More preferably, the ligand binding molecule is a growth factor, an antibody or antibody fragment to a growth factor, or an antibody or antibody fragment to a cell surface receptor. Alternatively, the ligand or targeting unit can comprise an antibody, antibody fragment (e.g., an F(ab′) 2 fragment) or analogues thereof (e.g., single chain antibodies) which bind a cell surface component (see e.g., Chen et al., 1994; Ferkol et al., 1998; Rojanasalcul et al., 1994), typically a receptor, which mediates internalization of bound ligands by endocytosis. Such antibodies can be produced by standard procedures then bound to the conjugate and be used in vitro or in vivo to selectively deliver said conjugates to target cells. The conjugate is stable and soluble in physiological fluids and can be administered in vivo where it is taken up by the target cell via the surface-structure-mediated endocytotic pathway.

The targeting moiety typically performs at least two functions:

1) It helps to bind the conjugate to target tissue creating an accumulation effect of the conjugate in and near the pathology.

2) It binds to a component on the surface of a target cell so that the carrier complex is internalized by the cell.

[symbol] can also be a component of a biological organism such as a virus, cells (e.g., mammalian, bacterial, protozoan).

Aside from the already discussed strategies to covalently bind targeting moieties to the photosensitizer-conjugate, strategies for the non-covalent tethering of such units include but are not limited to hydrogen bonding interactions, hydrophobic, and electrostatic interactions which can be used alone or in any combination. For instance, a conjugate containing biotin moieties can be tethered to a biotinylated antibody through avidin or streptavidin.

As it is mentioned above, a further object of the invention accordingly provides a pharmaceutically acceptable composition. comprising a compound or a complex according to this invention, together with at least one pharmaceutical carrier or excipient. It will be apparent to persons skilled in the art that the concentrations of the compounds of the invention depend upon the nature of the compound, the composition, the mode of administration and the patient and may be varied of adjusted to choice. For topical application, e.g. concentration ranges from 0.05 to 50% (w/w) are suitable, more preferentially from 0.1 to 20%. Alternatively, for systemic application drug doses of 0.05 mg/kg body weight to 1000 mg/kg body weight of photosensitizer equivalents, more preferentially 0.1 to 100 mg/kg, are appropriate.

### III. Photosensitizers and use Thereof

It is envisioned that virtually any photosensitizer may be used with the present invention. Photosensitizers include HpD as well as more modern photosensitizers. Various photosensitizers have been described, including improvements on HpD per se such as disclosed in the U.S. Pat. Nos. 5,028,621; 4,866,168; 4,649,151; and 5,438,071. Furthermore, pheophorbides as disclosed in the U.S. Pat. Nos. 5,198,460; 5,002,962; and 5,093,349, bacteriochlorins in the U.S. Pat. Nos. 5,173,504, and 5,171,747. The use of phthalocyanine dyes in PCT is described in the U.S. Pat. No. 5,166,197 and green porphyrins are disclosed in the U.S. Pat. Nos. 4,883,790; 4,920,143; and 5,171,749. Conjugates of chlorophyll and bacteriochlorophylls are disclosed in U.S. Pat. No. 6,147,195. The content of these patents are incorporated herein as reference.

Methods according to this invention employ, in general, several distinct steps. Firstly, a compound, complex or composition according to this invention is applied, preferentially to a [symbol] . Following administration the area of interest is exposed to light in order to achieve a photochemotherapeutic effect. The time period between administration and irradiation, will depend among others on the nature of the compound, the composition, the form of administration and the subject. The inventors prefer time periods between 4 minutes and 168 hours, more preferentially between 15 minutes and 96 hours.

The irradiation will be performed using a continuous or pulsed light source with light doses ranging from 2-500 J/cm², the inventors prefer light doses between 5 and 200 J/cm². Thereby the light dose may be applied in one portion or several distinct portions.

It will be understood from persons skilled in the art, that the wavelength of light used for the irradiation, must be selected from at least one of the absorptions bands of the photosensitizing moiety of such conjugates in its phototoxically active configuration. Conventionally, when porphyrins are used as photosensitizing moieties, they are irradiated with wavelength in the region between 350 and 660 nm. For chlorines this range should be extended to 700 nm, while phthalocyanines an even larger range (350 to 800 nm) is suitable.

It should be mentioned that particularly the highest and lowest absorption bands of the particular photosensitizing moiety are of interest. By this, the inventors mean that wavelengths in the red region of the spectrum are particularly useful for treating bulky or deeper lying lesions and disease in the retina or the subretina, as well as vascular lesions. Wavelength in the blue region of the visible spectrum are useful for treating superficial lesions thus preventing side effects including pain, stenosis, occlusion, or necrosis in muscle tissue. However, superficial lesions can also be treated with red or green light.

TABLE 3
Some exemplary photosensitizers with selected wavelength regions
with respect to methods according to this invention.

| | Blue Region | Green Region | Red Region |
|---|---|---|---|
| Name | [nm] | [nm] | [nm] |
| Hematoporphyrin | 380-420 | 490-520 | 600-670 |
| Derivative (HPD) | (405) | (502) | (630) |
| Photofrin II | 380-420 | 490-520 | 600-670 |
| (PII) | (405) | (502) | (630) |
| Tetra(m-hydroxy- | 400-450 | 500-560 | 600-680 |
| phenyl)chlorin (mTHPC) | (420) | (520) | (652) |
| Benzoporphyrin Derivative | 400-460 | | 600-670 |
| Mono Acid Ring | (430) | | (630) |
| (BPD-MA) | | | 670-720 |
| | | | (690) |
| Zinc-Phthalocyanin | 320-400 | | 580-630 |
| (ZnPC) | (343) | | (607) |
| | | | 650-700 |
| | | | (671) |
| Protoporphyrin IX | 380-440 | | 600-680 |
| | (405) | | (635) |
| Chlorin e6 | 380-440 | | 600-690 |
| | (410) | | (662) |
| AlS4Pc | 320-400 | | 580-630 |
| | (343) | | (607) |
| | | | 650-700 |
| | | | (671) |
| Texaphyrins | 400-500 | | 690-780 |
| | 450 | | (732) |
| Hypericin | 400-500 | 520-600 | 570-650 |
| | (475) | (550) | (592) |
| Pheophorbide a | 350-450 | | 600-720 |
| | (400) | | (670) |

The wavelength in brackets describe the maxima of the particular absorption band with a deviation of ±5 nm. The table shows only some examples for useful photosensitizing moieties and should not be understood as limitation.

Methods of irradiation of different area of the body and methods to bring light to the internal body cavities from light sources including lamps, laser, and light emitting diodes are well known in the art and described in detail in References and it is obvious to persons skilled in the art, that alternatively traiisdennal irradiation can be performed.

### IV. Treatment of Diseases

The present invention includes methods, using compounds or complexes according to the invention or any pharmaceutically acceptable composition thereof for therapeutic purposes, preferentially photochemotherapeutic purposes. Diseases or disorders, which may be treated according to the present invention include any malignant, pre-malignant and non-malignant abnormalities responsive to photochemotherapy, including, but not limited to, tumors or other growth, skin disorders such as psoriasis, skin cancer, or actinic keratosis, and other diseases or infections, e.g. bacterial, viral or fungal infections. Methods according to this invention are particularly suited when the disease is located in areas of the body that are easily accessible to light, such as internal or external body surfaces. These surfaces include, e.g. the skin and all other epithelial and serosal surfaces, including for example mucosa, the linings of organs, e.g. the respiratory, gastro-intestinal and genito-urinary tracts, and glands, and vesicles.

In addition to the skin, such surfaces include for example the lining of the vagina, the endometrium, the peritoneum, the urothelium, and the synovium. Such surfaces may also include cavities formed in the body following excisions or incisions of diseased areas, e.g. brain cavities. Exemplary surfaces using methods according to this invention are listed in Table 2:

TABLE 2
List of some exemplary body surfaces

Skin
Conjunctiva
Linings of the mouth, pharynx, and larynx
Linings of the oesophagus, stomach, intestines, and intestinal
appendages
Linings of the rectum and the anal canal
Linings of the nasal passages, nasal sinuses, nasopharynx
Linings of the trachea, bronchi, and bronchioles
Linings of the ureters, urinary bladder, and urethra
Linings of the vagina, uterine cervix, and uterus
Parietal and visceral pleura
Linings of the peritoneal and pelvic cavities
Dura mater and meninges
Any tumor in solid tissues that can be made accessible to
photoactivating light

For persons skilled in the art of PCT, it will be apparent that methods are not only limited to either malignant, or pre-malignant or non-malignant abnormalities which are present at body surfaces. For persons skilled in the art of PCT, it will also be apparent that [symbol] according to this invention may also be suitable for the treatment of angiogenesis associated diseases, when the target tissue is vascular endothelial tissue. Typical examples include, but are not limited to an abnormal vascular wall of a tumor, a solid tumor, a tumor of a head, a tumor of a neck, a tumor of a gastrointestinal tract, a tumor of a liver, a tumor of a breast, a tumor of a prostate, a tumors of a lung, a nonsolid tumor, malignant cells of one of a hematopoietic tissue and a lymphoid tissue, lesions in a vascular system, a diseased bone marrow, and diseased cells in which the disease is one of an autoimmune and an inflammatory, such as rheumatoid arthritis disease or chorioallantoic neovascularization associated with age-related macular degeneration. In yet a further method of the present invention, the target tissue is a lesion in a vascular system. It is contemplated that the target tissue is a lesion of a type selected from the group consisting of atherosclerotic lesions, arteriovenous malformations, aneurysms, and venous lesions.

Methods according to this invention may also be used for cosmetic purposes, hair removal, depilation, removing varicoses, the treatment of acne, skin rejuvenation etc.

The present invention may also be useful for the treatment of Protista and parasitic origin, as defined above, particularly acne, malaria and other parasites or lesions resulting from parasites.

The term “parasite” includes parasites of humans and other animals, including parasitic protozoa (both intracellular and extracellular), parasitic worms (nematodes, trematodes, and cestodes) and parasitic ectoparasites (insects and mites).

The parasitic Protozoa include: malarial parasites which may affect humans and/or other animals such as:

| | | |
|---|---|---|
| Plasmodium falciparum | Plasmodium ovale | Plasmodium malaria |
| Plasmodium vivax - | leishmanial parasites of | Leishmania tropica |
| leishmanial parasites of | humans | |
| humans and or other | | |
| animals | | |
| Leishmania major | Leishmania aethiopica | Leishmania brasiliensis |
| Leishmania guyanensis | Leishmania panamenis | Leishmania peruviana |
| Leishmania mexicana | Leishmania amazonensis | Leishmania pifanoi |
| Leishmania garnhami | Leishmania donovani | Leishmania infantum |
| Leishmania chagasi - | trypanosomal parasites of | Trypanosoma cruzi |
| trypanosomal parasites of | humans | |
| humans and/or other | | |
| animals | | |
| Trypanosoma brucei | Trypanosoma brucei | amoebic parasites of humans |
| gambiense | rhodesiense - amoebic | |
| | parasites of humans and/or | |
| | other animals | |
| Entamoeba histolytica | Naeglaria species | Acanthamoeba species |
| Dientamoeba fragilis - | miscellaneous protozoan | Toxoplasma gondii |
| miscellaneous protozoan | parasites of humans | |
| parasites of humans or | | |
| other animals | | |
| Pneumocystis carinii | Babesia microti | Isospora belli |
| Cryptosporidium | Cyclospora species | Giardia lamblia |
| Balantidium coli | Blastocystis hominis | Microsporidia species |
| Sarcocystis species | Some of these miscellaneous | parasitic nematodes in |
| | protozoa cause self-limiting | humans and/or other animals |
| | disease in normal people, but | |
| | serious problems in HIV | |
| | patients. | |
| parasitic nematodes in | filarial nematodes | Wuchereria bancrofti |
| humans | | |
| Brugia malayi | Brugia timori | Onchocerca volvulus |
| Loa loa | Tetrapetalonema perstans | Tetrapetalonema streptocerca |
| Mansonella ozzardi | Dirofilaria immitis | Dirofilaria tenuis |
| Dirofilaria repens - | Ascaris lumbricoides | Necator americanus |
| intestinal nematodes | (roundworm) | (hookworm) |
| Ancylostoma duodenale | Strongyloides stercoralis | Enterobius vermicularis |
| (hookworm) | (threadworm) | (pinworm) |
| Trichuris trichiura | Trichostrongylus species | Capillaria philippinensis - |
| (whipworm) | | tissue nematodes |
| Trichinella spiralis | Anasakis species | Pseudoterranova species |
| Dracunculus medinensis - | parasitic trematodes in | Schistosoma mansoni |
| parasitic trematodes in | humans | |
| humans and/or other | | |
| animals | | |
| Schistosoma haematobium | Schistosoma japonicum | Clonorchis sinensis |
| Paragonimus species | Opisthorchis species | Fasciola hepatica |
| Metagonimus yokogawai | Heterophyes heterophyes | Fasciolopis buski - parasitic |
| | | cestodes in humans and/& |
| | | other animals |
| parasitic cestodes in | Taenia saginata | Taenia solium |
| humans | | |
| Hymenolepis species | Diphyllobothrium species | Spirometra species |
| Echinococcus species | | |

It will be understood that methods using compounds according to this invention may also be usefull for sterilization in food industry and agriculture.

### EXAMPLES

The following examples illustrate several embodiments of the present invention. They are not intended to restrict the invention, which is not limited to specific embodiments, polymers, biocompatibilizing molecules, targets, photosensitizer, fluorophore, or quenching moieties. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

Preparation of pheophorbide a-NHS ester: To a solution of pheophorbide a (Frontier Scientific) (300 mg, 0.506 mmol) in CH₂Cl₂ (95 mL) were added EDC (1.7 equiv, 0.165 g), N-bydroxysuccinimide (1.7 equiv, 0.10 g) and DMAP (0.4 equiv, 24 mg) and the mixture stirred [symbol] the dark. Solvent was removed under reduced pressure and the purified by flash chromatography on a silica gel column. The product was obtained as a dark solid (230 mg).

Preparation of a photosensitizer-polymer conjugate comprised of a poly-L-lysine backbone with 5% loading of pheophorbide a via N-epsilon amide bonds: in a small vial fitted with a strong magnetic stirrer was dissolved PL-HBr (8.0 mg, 3.23×10⁻⁴ mmol) in dry DMSO (1.5 mL) then was added DIPEA (6 equiv. per NH₂ side chain, 30 mg) and dry DMF (0.8 mL). This solution was stirred for 10 min. before adding dropwise and under vigorous stirring pheophorbide a-NHS ester (5% equiv. per NH₂ side chain, 1.31 mg, 0.001905 mmol) in DMF (1.0 mL). The resulting solution was stirred in the dark for 1 h, then solvent was removed under reduced pressure. The resulting oil (DMSO+reaction products) was dissolved in water to make 5.3 mL of solution and the aqueous phase extracted 2× with CH₂Cl₂ (7.0 mL) to remove unreacted pheophorbide. The aqueous phase was then filtered and the product purified by size exclusion chromatography using a sephacryl™ S-100 (Amersham Biosciences) column and 35:65:0.05 acetonitrile/water/TFA as eluent. The fraction containing the product was lyophilized to yield the desired product as a green fluffy solid.

### Example 2

Preparation of a photosensitizer-polyrner conjugate comprised of a poly-L-lysine backbone with 10% loading of pheophorbide a via N-epsilon amide bonds: in a small vial fitted with a strong magnetic stirrer was dissolved PL-HBr (8.0 mg, 3.23×10⁴ mmol) in dry DMSO (1.5 mL) then was added DIPEA (6 equiv. per NH₂ side chain, 30 mg) and dry DMF (0.8 mL). This solution was stirred for 10 min. before adding dropwise and under vigorous stirring pheophorbide a-NHS ester (0.10 equiv. per NH₂ side chain, 2.62 mg, 0.00381 mmol) in DMF (1.0 mL). The resulting solution was stirred in the dark for 1 h, solvent was then removed under reduced pressure. The resulting oil (DMSO+reaction products) was dissolved in water to make 5.3 mL of solution and the aqueous phase extracted 2× with CH₂Cl₂ (7.0 mL) to remove unreacted pheophorbide. The aqueous phase was then filtered and the product purified by size exclusion chromatography using sephacryl™ S-100 (Amersham Biosciences) column and 35:65:0.05 acetonitrile/water/TFA as eluent. The fraction containing the product was lyophilized to yield the desired product as a green fluffy solid.

### Example 3

Preparation of a photosensitizer-polymer conjugate comprised of a poly-L-lysine backbone with 15% loading of pheophorbide a via N-epsilon amide bonds: in a small vial fitted with a strong magnetic stirrer was dissolved PL-HBr (8.0 mg, 3.23×10⁻⁴ mmol) in dry DMSO (1.5 mL) then was added DIPEA (6 equiv. per NH₂ side chain, 30 mg) and dry DMF (0.8 mL). This solution was stirred for 10 min. before adding dropwise. and under vigorous stirring pheophorbide a-NHS ester (0.15 equiv. per NH₂ side chain, 3.94 mg, 0.00572 mmol) in DMF (1.0 mL). The resulting solution was stirred in the dark for 1 h, then solvent was removed under reduced pressure. The resulting oil (DMSO+reaction products) was dissolved in water to make. 5.3 mL of solution and the aqueous phase extracted 2× with CH₂Cl₂ (7.0 mL) to remove unreacted pheophorbide. The aqueous phase was then filtered and the product purified by size exclusion chromatography using sephacryl™ S-100 (Amersham Biosciences) column and 35:65:0.05 acetonitrile/water/TFA as eluent. The fraction containing the product was lyophilized to yield the desired product as a green fluffy solid.

### Example 4

Preparation of a photosensitizer-polymer conjugate comprised of a poly-L-lysine backbone with 25% loading of pheophorbide a via epsilon N-amide bonds: in a small vial fitted with a strong magnetic stirrer was dissolved PL-HBr (8.0 mg, 3.23×10⁻⁴ mmol) in dry DMSO (1.5 mL).

This solution was stirred for 10 min. before adding dropwise and under vigorous stirring pheophorbide a-NHS ester (0.25 equiv. per NH₂ side chain, 6.60 mg, 0.00953 mmol) in DMF (1.0 mL). The resulting solution was stirred in the dark for 1 h, then solvent was removed under reduced pressure. The resulting oil (DMSO+reaction products) was dissolved in water to make 5.3 mL of solution and the aqueous phase extracted 2× with CH₂Cl₂ (7.0 mL) to remove unreacted pheophorbide. The aqueous phase was then filtered and the product purified by size exclusion chromatography using sephacryl™ S-100 (Amersham Biosciences) column and 35:65:0.05 acetonitrile/water/TFA as eluent. The fraction containing the product was lyophilized to yield the desired product as a green fluffy solid.

### Example 5

Preparation of a photosensitizer-polyiner conjugate comprised of a poly-L-lysine backbone with 25% loading of pheophorbide a via a cathepsin D cleavable linker and 20% loading of mPEG through permissible epsilon N-amide bonds: in a small vial fitted with a strong magnetic stirrer was dissolved PL-HBr (8.0 mg, 3.23×10⁻⁴ mmol) in dry DMSO (1.5 mL) then was added DIPEA (6.0 equiv. per NH₂ side chain, 30 mg) and dry DMF (0.8 mL). This solution was stirred for 10 min. before adding dropwise and under vigorous stirring rnPEG-NHS activated ester (2 kDa, Nektar Therapeutics, 0.2 equiv. per epsilon NH₂ groups of PL, 38.3 mg, 0.00766 nmol) in DMF (0.50 mL). The resulting solution was stirred in the dark for 16 h, then cooled to 0° C. and under vigorous stirring was added dropwise iodoacetic anhydride (1.0 equiv. per epsilon NH₂ group of PL, 0.0383 mnol, 13.5 mg) in DMF (0.5 mL) and the mixture allowed to react for 2 h after the addition. Solvent was removed under reduced pressure. The resulting oil (DMSO+reaction products) was dissolved in water to make 5.3 mL of solution and filtered. The crude product was purified by size exclusion chromatography using a sephacryl™ S-100 (Amersham Biosciences) column and 100:0.025 water/TFA as eluent. The fraction containing the product was lyophilized to yield the desired intermediate product as a white fluffy solid. The product obtained in the previous step was dissolved in a NaHCO₃ buffer (8.0 mL) and under continuous stirring was added dropwise pheophorbide a-NH-Gly-Pro-Ile-Cys(Et)-Phe-Phe-Arg-Leu-Gly-Cys-OH-TFA (0.25 equiv. per epsilon NH₂ group in PL, 17.5 mg) in DMF (5.0 mL). The mixture was allowed to react for 16 h then was added cysteine (10 equiv. per epsilon NH₂ group, 46.4 mg) and allowed to react for 8 additional hours. The product was then purified by size exclusion chromatography as before and lyophilized to obtain a green fluffy solid.

For the preparation of pheophorbide a-NH-Gly-Pro-Ile-Cys(Et)-Phe-Phe-Arg-Leu-Gly-Cys-OH-TFA: The peptide was manually assembled on the solid phase using the Fmoc strategy on a HN-Cys(Trt)-2-chlorotrityl resin (Bachem). Once the peptide reached the desired length (Fmoc-NH-Gly-Pro-Ile-Cys(Et)-Phe-Phe-Arg(Pbf)-Leu-Gly-Cys(Trt)-2-chlorotrityl resin), it was Fmoc deprotected using a standard protocol (20% piperidine in DMF) and coupled with 1.3 equiv. of pheophorbide a-NHS ester overnight. The peptide-pheophorbide a conjugate was cleaved from the solid-phase and purified by reverse-phase HPLC on a C-18 column (Macherey-Nagel). Product was obtained as a greenish solid.

### Example 6

Preparation of a control non-activatible photosensitizer-polymer conjugate comprised of a poly-L-lysine backbone with 25% loading of pheophorbide a via a pennutated cathepsin D non-cleavable linker and 20% loading of mPEG through permissible epsilon N-amide bonds: in a small vial fitted with a strong magnetic stirrer was dissolved PL-HBr (8.0 mg, 3.23×10⁻⁴ mmol) in dry DMSO (1.5 mL) then was added DIPEA (6.0 equiv. per NH₂ side chain, 30 mg) and dry DMF (0.8 mL). This solution was stirred for 10 min. before adding dropwise and under vigorous stirring mPEG-NHS activated ester (2 kDa, Nektar Therapeutics, 0.2 equiv. per epsilon NH₂ groups of PL, 38.3 mg, 0.00766 mmol) in DMF (0.50 mL). The resulting solution was stirred in the dark for 16 h, then cooled to 0° C. and under vigorous stirring was added dropwise iodoacetic anhydride (1.0 equiv. per epsilon NH₂ group of PL, 0.0383 mmol, 13.5 mg) in DMF (0.5 mL) and the mixture allowed to react for 2 h after the addition. Solvent was removed under reduced pressure. The resulting oil (DMSO+reaction products) was dissolved in water to make 5.3 mL of solution and filtered. The crude product was purified by size exclusion chromatography using a sephacryl™ S-100 (Amersham Biosciences) column and 100:0.025 water/TFA as eluent. The fraction containing the product was lyophilized to yield the desired intermediate product as a white fluffy solid. The product obtained in the previous step was dissolved in a NaHCO₃ buffer (8.0 mL) and under continuous stirring was added dropwise pheophorbide a-NH-Gly-Cys-Pro-Ile-Cys(Et)-Phe-Phe-Arg-Leu-Gly-OH-TFA (0.25 equiv. per epsilon NH₂ group in PL, 17.5 mg) in DMF (5.0 mL). The mixture was allowed to react for 16 h then was added cysteine (10 equiv. per epsilon NH₂ group, 46.4 mg) and allowed to react for 8 additional hours. The product was then purified by size exclusion chromatography as before and lyophilized to obtain a green fluffy solid.

For the preparation of a near-infrared probe reported by Weissleder et al. (2003), the inventors followed a literature procedure (Ching-Hsuan et al., 1999)

### Example 7

In order to investigate the fluorescence behavior of first generation pheophorbide a-PL conjugates upon enzymatic degradation (trypsin) with respect to pheophorbide a loading, the inventors looked at the kinetics of the degradation versus the apparent increased in fluorescence.

[symbol] generation pheophorbide a-PL stock solutions: dissolve 1.0 mg of the corresponding conjugate in 1:3 DMSO/H₂O to make 5.0 mL of solution. These stock solutions were placed in the refrigerator and protected from light prior to use.

Fluorescence measurements: 0.2 mL of the corresponding stock solution was mixed with 2.0 mL of trypsin-EDTA solution containing 0.5g of porcine trypsin, 0.2 g of EDTA, and 4.0 Na/L HBSS (Sigma) and the mixture quickly stirred and incubated in the dark at 37° C. Fluorescence (using excitation at 390 nm and emission at 670 nm) was followed overtime by sampling 0.2 mL of reaction mixture in 0.6 mL of DMSO. The fluorescence at time equal zero was determined by adding together the fluorescence of the enzyme and pheophorbide a-PL conjugate. Thus, the enzyme fluorescence was determined by diluting 0.2 mL of PBS saline buffered solution with 2.0 mL of trypsin-EDTA then sampling 0.2 mL of this solution in 0.6 mL of DMSO. Similarly, the baseline pheophorbide a-PL fluorescence was determined by diluting 0.2 mL of the corresponding stock solution with 2.0 mL of PBS saline buffered solution then sampling 0.2 mL of this solution in 0.6 mL of DMSO.

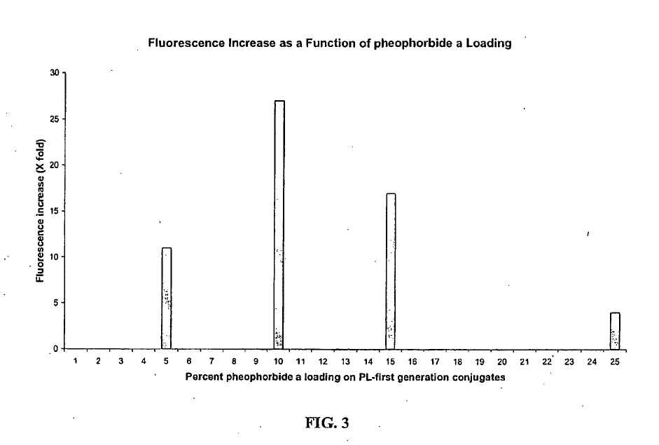

The results from this investigation revealed that the “maximum” increased in fluorescence for the 5%, 10%, 15%, and 25% loaded pheophorbide a-poly-(L)-lysine conjugates was achieved at times equal to 4 min, 8 min, 13 min, and 40 min respectively. FIG. 3 shows the “maximum” relative increase in fluorescence for each of the first generation probes tested. The respective fluorescence increase values for the 5%, 10%, 15% and 25% loaded probes are 11, 27, 17, and 4. Thus the maximum fluorescence increase (27 fold) was attained with the 10% loaded pheophorbide a-PL conjugate.

### Example 8

The photosensitizing behavior of first generation pheophorbide a-conjugates was investigated by measuring their ability to generate ROS in solution. These experiments were carried out with the ROS sensitive probe, dihydro-rhodamine 123. (Seung-Cheol et al., 2005.)

ROS measurements using dihydro-rhodarnine 123: Solution 1: 0.05 mL of the corresponding first generation pheophorbide a-PL stock solution was combined with 1.0 mL of trypsin-EDTA solution containing 0.5 g of porcine trypsin, 0.2 g of EDTA, and 4.0 Na/L HBSS (Sigma) and the mixture quickly stirred and incubated in the dark at 37° C. for the indicated amount of time (corresponding to 5 min, 8 min, 13 min., and 40 min. for the 5%, 10%, 15% and 25% loaded conjugates respectively). Solution 2: similarly, 0.05 mL of the [symbol] combined with 1.0 mL PBS saline buffer solution and the mixture quickly stirred and incubated in the dark at 37° C. for the indicated amount of time (corresponding to 5 min., 8 min., 13 min., and 40 min. for the 5%, 10%, 15% and 25% loaded probes respectively). At the end of the incubation period were added 1.0 mL of DMSO and 40 μL of an 80 mM DHR123 solution to each of two solutions. Then, 0.5 mL aliquots of each of the resulting solutions were simultaneously irradiated with white light for 2 min using two adjacent wells of a 24 well cell culture plate. The remainder of the solution derived from solution 2 was kept in the dark and used to measure the baseline fluorescence. Each fluorescence measurement (using excitation at 495 nm and emission at 535 nm) was made by taking 0.1 mL aliquots of the corresponding solution and diluting with 0.6 mL of DMSO.

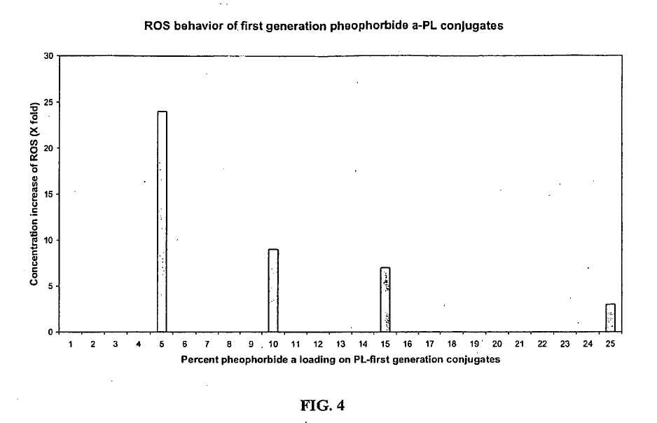

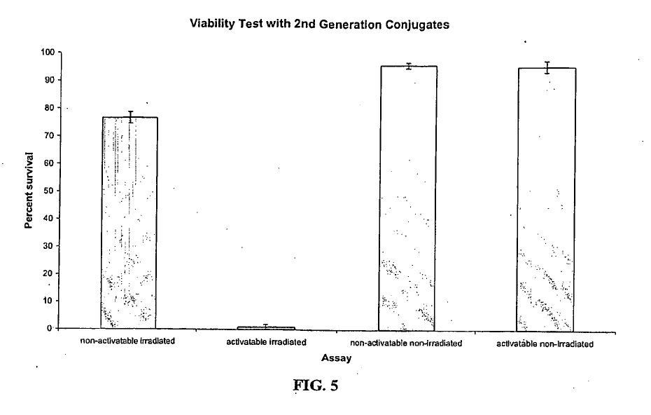

The surprising results shown in FIG. 4 indicate that indeed said conjugates become phototoxically activated by trypsin. The results also indicate that the fluorescence properties of these conjugates does not necessarily match with their photosensitizing behavior (compare FIGS. 1 and 2). Thus, the maximum increase in fluorescence was achieved with the 10% pheophorbide a-PL conjugate, while the maximum concentration increase of ROS was achieved with the 5% pheophorbide a-PL conjugate.

### Example 9

In order to test the phototoxicity in vitro of the second generation pheophorbide a-poly-L-lysine conjugates, exemplified in example 6, Cath D-1 cells were treated with said pheophorbide a-PL conjugate, with the conjugate and light, with the non-activatable conjugate, and with non-activatable conjugate and light. The therapeutic outcome was assessed by an MTT assay. In addition, the inventors also tested the phototoxicity in vitro of a particular probe described by Weissleder and coworkers (Ching-Hsuan et al., 1999).

1. Cells

The Cath D-1 cell line was prepared according to Liaudet et al. (1995). Cells were cultured in 24-well multiwell dishes using Dulbecco's Modified Minimum Essential Medium (DMEM) with Earle's salts containing 10% fetal calf serum (FCS), 100 U/ml penicillin 0.2 mg/ml streptomycin, 0.2 % glycine at 37° C. in 5% CO₂, 95% air in a humidified atmosphere. After confluence, the cells were washed two times with HBSS.

2. Treatment

Cells were incubated with the second generation pheophorbide a-PL conjugates (examples 5 and 6) at 3 μM concentrations. Incubation with the conjugates was performed for 60 minutes and cells were then irradiated for 15 min at 410 nm with a light dose of 5 J/cm² (in the case of the near-infrared probe by Weissleder (2003), the inventors irradiated for 15 min at 680 nm with the same light dose). The cells were rinsed with HBSS and incubated in the dark with DMEM for twenty-four hours. The viability test was performed using an MTT assay.

3. Determination of Cell Viability

The cell viability was tested by means of an MTT assay. This technique allows quantification of cell survival after cytotoxic insult by testing the enzymatic actitivity of the mitochondria. It is based on the reduction of the water-soluble tetrazoliurn salt to a purple, insoluble formazan derivative by the mitochondrial enzyme dehydrogenase. This enzymatic function is only present in living, metabolically active cells. The optical density of the product was quantified by its absorption at 540 nm using a Safire plate reader. MTT, 0.1%, was added to each well (200 μL) 24 hours after irradiation and incubated for 3 hours at 37° C., then was added DMSO (800 μL per well) and incubated for an additional hour at 37° C. before measuring the absorbance. The absorption of the solution in each well was determined by using the plate reader at 540 nm. Absorbance of the solution from treated cells was divided by the absorption of the solution from the control cell plates to calculate the fraction of surviving cells.

4. Results

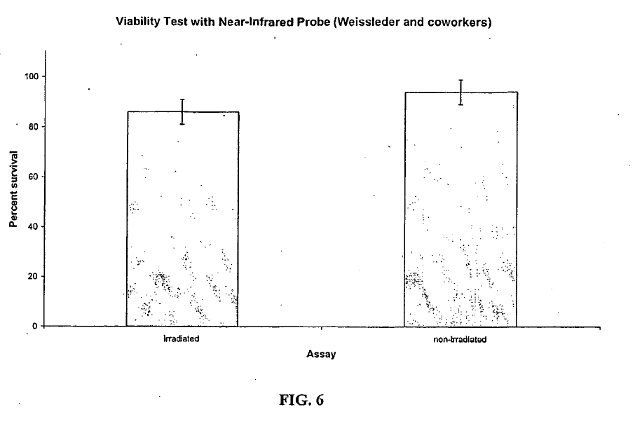

FIG. 5 shows the results of the viability test. Clearly, the data show that the pheophorbide a-PL activatible conjugate (example 5) indeed becomes considerable more phototoxic in the presence of cathepsin D positive cells. This phototoxicity is greatly inhibited by using the non-activatable pheophorbide a-PL conjugate (example 6).

### Example 10

| | | | fluorescence | fluorescence |
|---|---|---|---|---|
| | | relative | intensity (AU) | increase |
| | percent | water | at equimolar | (Xfold) upon |
| | solubil- | solubil- | concentra- | enzymatic |
| Solubilizer moiety | izer | ity mM | tion* ×10⁶ | degradation |
| none | — | 1.3 | 3.13 | 11 |
| 2-(N,N,N-Trimethyl- | 25 | 0.3 | 1.2 | 11 |
| ammonium)ethanoic | | | | |
| acid | | | | |
| 1-Methyl nicotinamide | 25 | >10 | 1.0 | 11 |
| 1-Methyl nicotinamide | 85 | >10 | 1.3 | 0.2 |
| Monosuccinamide | 85 | 0.9 | 1.4 | 0.2 |

Data for 15% loaded pheophorbide a-PL conjugates.
*Equimolar concentration with respect to the photosensitizer as determined by absorbance at 675 nm.

General procedure for the preparation of photosensitizer-poly(L-lysine) conjugates carrying solubilizing/enzymatic protecting moieties: (A) 1-methyl nicotinaniide or (B) monosuccinamide moieties: To a solution of poly(L-lysine) (25 KDa or 7.5 KDa) (8.0 mg, 3.83×10⁻⁵ moles of epsilon NH₂ functions) in anhydrous DMSO (0.84 mL) was added DIPEA (3.0 equiv per epsilon NH₂, 14.8 mg); thereafter, the activated photosensitizer-NHS in DMSO (7 mg/mL) was added under vigorous stirring. The progress of this quantitative coupling reaction was monitored by analytical HPLC using a C18 column (Macharey Nagel) and water/acetonitrile/TFA (50:50:0.001) as eluent. At this point, either N-methylnicotinic acid NHS ester iodide (N-succinimidyl (1-methyl-3-pyridinio)formate iodide) (for the preparation of A)) in DMSO (12 mg/mL) or succinic anhydride (for the preparation of B) in DMSO (12 mg/mL) was added dropwise with vigorous stirring and allowed to react for two additional hours. The reaction mixture was then quenched by adding water (3.0 mL) and either TFA to pH 2-3 for A or conc. NH₃ to pH 9 for B. The resulting solution was filtered and purified by size exclusion chromatography (SEC) using a sephacryl™ S-100 (Amersham Biosciences) column and either 35:65:0.00025 acetonitrile/water/TFA for A or 35:65:0.00025 acetonitrile/water/NH₃ for B as eluent. The fraction containing tie product was lyophilized to yield the desired product as a green solid.

### Example 11

General procedure for the preparation of second generation photosensitizer-poly(L-lysine) conjugates—cleavable linker has trypsin sensitive sequence Gly-Thr-Phe-Arg-Ser-Ala-Gly (SEQ ID NO:1): To a solution of poly(L-lysine) (25 KDa or 7.5 KDa) (8.0 mg, 3.83×10⁻⁵ moles of epsilon NH₂ functions), pheophorbide a-Gly-Thr-Phe-Arg-Ser-Ala-Gly·TFA (0.25 equiv per NH₂ side chains, 13.2 mg), and HATU (1.2 equiv per pheophorbide a-peptide unit, 4.4 mg) in anhydrous DMSO (1.2 mL) was added DIPEA (4.0 equiv per epsilon NH₂, 19.8 mg) and the reaction stirred under argon overnight. The progress of the coupling reaction was monitored by analytical HPLC using a C18 column (Macharey Nagel) and water/acetonitrile/TFA (50:50:0.001) as eluent (coupling efficiency was found to be between 90-95%). At this point, N-methylnicotinic acid NHS ester iodide (N-succinimidyl (1-methyl-3-pyridinio)formate iodide) (0.6 equiv per NH₂ side chains, 8.3 mg) in DMSO (0.7 mL) was added dropwise with vigorous stirring and allowed to react for two additional hours. The reaction mixture was then quenched by adding water (5.0 mL) and TFA to pH 2-3. The resulting solution was filtered then purified by size exclusion chromatography (SEC) using a sephacryl™ S-100 (Amersham Biosciences) column and 30:70:0.00025 acetonitrile/water/TFA as eluent. The fraction containing the product was lyophilized to yield the desired product as a green solid.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

U.S. Pat. No. 4,649,151

U.S. Pat. No. 4,866,168

U.S. Pat. No. 4,883,790

U.S. Pat. No. 4,920,143

U.S. Pat. No. 5,002,962

U.S. Pat. No. 5,028,621

U.S. Pat. No. 5,093,349

U.S. Pat. No. 5,166,197

U.S. Pat. No. 5,171,747

U.S. Pat. No. 5,171,749

U.S. Pat. No. 5,173,504

U.S. Pat. No. 5,198,460

U.S. Pat. No. 5,438,071

U.S. Pat. No. 6,147,195

Agrawal and Zamecnik, Nucleic Acids Res., 18(18):5419-5423, 1990.

Akhlynina et al., Cancer Res., 55(5):1014-1019, 1995.

Baldwin et al., Biochemistry, 29:5509-5515, 1990.

Beaucage, Methods Mol Biol., 20:33-61, 1993.

Biel, Laryngoscope, 108(9):1259-1268, 1998.

Bigelow et al., J. Microscopy, 215(1):24-33, 2004.

Birchler et al., J. Immunol. Methods, 231(1-2):239-248, 1999.

Birchler et al., Nat. Biotechnol., 17(10):984-988, 1999.

Boal et al., Nucleic Acids Res., 24(15):3115-3117, 1996.

Breinbauer and Koehn, Chem. Bio. Chem., 4(11):1147-1149, 2003.

Bressler and Bressler, Invest. Ophthalinol. Vis. Sci., 41(3):624-628, 2000.

Chen et al., FEBS Letters, 338:167-169, 1994.

Ching-Hsuan et al., Bioconjugate Chemistry, 10(5);892-896, 1999.

Coussens et al., Science, 295:2387-2392, 2002

De Rosa and Bentley, Pharm Res., 17(12):1447-1455, 2000.

Del Governatore et al., Cancer Res., 60(15):4200-4205, 2000.

Dougherty et al., J. Natl. Cancer Inst., 90(12):889-905, 1998.

Ell and Gossner, Cancer Res., 155:175-181, 2000.

Evans and Kargman, Current Pharm. Design, 10(6):627-634, 2004.

Ferkol et al., J. Clin. Invest., 92:2394-2400, 1993.

Fukuda et al., Int. J Biochem. Cell Biology, 37(2):272-276, 2005.

Funovics et al., Analytical and Bioanalytical Chemistry, 377(6):956-963, 2003.

Funovics et al., Bioanalytical Chemistry, 377(6):956-963, 2003

Gomer et al., Cancer Res., 39(1):146-151, 1979.

Haimovici et al., Curr. Eye Res., 16(2):83 -90, 1997.

Hamblin et al., J. Photochem. Photobiol. B, 26(1):45-56, 1994.

Hamblin et al., Photochem. Photobiol., 72, 2000.

Hochhaus and Larosse, Leukemia, 18(8):1321-1331, 2004.

Hoffman and Stayton, Fundl. Applns. Polymer Gels, 207:139-151, 2004.

Hsi et al., Drugs, 57(5):725-734, 1999.

James et al., Bioorg. Med. Chem. Lett, 9, 1999.

Koehn and Breinbauer, Angewandte Chemie, Intl. Ed., 43(24):3106-3116, 2004.

Kolb et al., Drug Discovery Today, 8(24):1128-1137, 2003.

Lange, In: Controlled drug delivery in photodynamic therapy and fluorescence-based diagnosis of cancer, Pogue and Mycek (eds.), Handbook of Fluorescence in Medicine Marcel Dekker, NY, 2002.

Levine et al., Comp. Biochem. Physiol., 72B:77-85, 1982.

Liaudet et al., Cell Growth Differ., 6(9):1045-1052, 1995.

Lindberg et al., Oncologica, 43(2):142-152, 2004.

Lopez et al., Advanced Drug Deliv. Rev., 56(1):77-94), 2004.

Mahmood and Weissleder, Molecular Cancer Therapeutics, 2(5):489-496, 2003.

Mattoussi et al., JALA, 9(1):28-32, 2004.

McGuire, Current Pharm. Design, 9(31):2593-2613, 2003.

McIntyre et al., Biochem. J., 377:617 628, 2004.

Means and Feeney, J. Food Biochem., 22(5):399-425, 1998.

Morris et al., Plant Molecular Biology, 24:673:77, 1994.

Nagae et al., J. Cardiovasc. Sug. (Torino), 39(6):709-715, 1998.

Nseyo, Urol. Clin. North Am., 19(3):591-599, 1992. [symbol] , Nucleic Acids Res., 20(19):5205-5214, 1992.

PCT Appln. WO 2004/004769

PCT Appln. WO 2005007631

PCT Appln. WO 2003082988

PCT Appln. WO 2002056670

PCT Appln. WO 2002000265

PCT Appln. WO 19958161

Pech et al., Baillieres Best Pract. Res. Clin. Gastroenterol., 15(2):267-284, 2001.

Pham et al., Bioconjugate Chemistry, 15(6):1403-1407, 2004.

Rau et al., Europ. J. Biochemistry, 268(11):3284-3295, 2001.

Rojanasakul et al., Pharmaceutical Res., 11(12):1731-1736, 1994.

Rosenkranz et al., Immunol. Cell Biol., 78(4):452-464, 2000.

Seung-Cheol et al., Biochem. Biophysical Res. Comm., 327(4):979-984, 2005.

Sheski and Mathur, Cancer Control, 7(1):35-44, 2000.

Spitzer and Krumholz, Obstet. GynecoL Clin. North Am., 18(3):649-659, 1991.

Sternberg and Dolphin, J. Clin. Laser Med. Suig., 11(5):233-241, 1993.

Tang and Agrawal, Nucleic Acids Res., 18(21):6461, 1990.

Veronese and Morpurgo, Farmaco, 54(8):497-516, 1999.

Vrouenraets et al., Cancer Res., 61(5):1970-1975, 2001.

Vrouenraets et al., Int. J. Cancer, 88(1):108-114, 2000.

Wainwright, J. Antimicrob Chemother., 42(1):13-28, 1998.

Wallace and Fraser, Amino Acids, 26(4):353-365, 2004.

Ward et al., Photochem. Photobiol., 35:803-808, 1982.

Wilbanks et al., J. Biol. Chem., 268:1226-1235, 1993.

Zhou et al., Proc. SPIE Intl. Soc. Optical Eng., 4955:505-512, 2003.

1. A pharmacologically acceptable photosensitizer conjugate comprising one or more photosensitizer moiety conjugated to a biocompatible polymer backbone, wherein the conjugate is enzyme-activatable to increase the activity of the photosensitizer.

2. The conjugate of claim 1, wherein the polymer backbone is enzyme degradable.

3. The conjugate of claim 1, wherein the polymer backbone is degradable by a peptidase, a glycolytic enzyme, an esterase, a trypsin, a cathepsin, lipoprotein lipase, lecithin:cholesterol acyltransferase, 26-hydroxylase, an enzyme that regulates disorders in metabolism of porphyrins and heme, lysyl hydroxylase, collagenase, lactase, trehalase, prostate specific antigen (PSA), a matrix metalloproteinase, a CMV protease, or a proteosome.

4. The conjugate of claim 3, wherein the polymer backbone is degradable by cathepsin D, cathepsin B or cathepsin H.

5. The conjugate of claim 1, wherein the polymer is further defined as a dimer, trimer, an oligomer, a copolymer, a block copolymer, or a crosslinked polymer.

6. The conjugate of claim 1, wherein the polymer comprises an oligonucleotide, polypeptide, a polysaccharide, a polyamide, a polylactide, a polyacrylamide, a polystyrene, a polyurethane, a polycarbonate or a polyester.

7. The conjugate of claim 1, wherein the polymer comprises polylysine, poly-L-lysine, poly-D-lysine, polyarginine, polyornithine, polyglutamic acid, a peptide comprising L and/or D amino acids, polyvinyl alcohol, polyacrylic acid, polymethacrylate, polyacrylamide, polyalkylcyanoacrylate, polyhydroxyacrylate, polysuccinimide, polysuccinic anhydride, poly(hydroxyethyl methacrylate) (HEMA), chitosan, polyhydroxybutanoates, polyglycolic acid, copolymers of polylactides and polyglycolic acids, or polyvinyl alcohol.

8. The conjugate of claim 1, wherein an enzyme-cleavable linker is conjugated to the polymer backbone.

9. The conjugate or claim 8, wherein the photosensitizer moiety or a quencher is conjugated to the enzyme-cleavable linker.

10. The conjugate of claim 8, wherein the enzyme-cleavable linker is a cathepsin D cleavable linker or an Epsilon N-amide bond.

11. The conjugate of claim 8, wherein the enzyme-cleavable linker is an amino acid sequence.

12. The conjugate of claim 8, wherein the amino acid sequence comprises Gly-Thr-Phe-Arg-Ser-Ala-Gly (SEQ ID NO:1).

13. The conjugate of claim 1, wherein the photosensitizer moiety is selected from the group consisting of chlorines, chlorophylls, coumarines, cyanines, fullerenes, metallophthalocyanines, metalloporphyrins, methylenporphyrins, naphthalimides, naphthalocyanines, nile blue, perylenequinones, phenols, pheophorbides, pheophyrins, phthalocyanines, porphycenes, porphyrins, psoralens, purpurins, quinines, retinols, rhodamines, thiophenes, verdins, xanthenes, and dimers and oligomers thereof.

14. The conjugate of claim 13, wherein the photosensitizer moiety is hematoporphyrin derivative (HPD), photofrin II (PII), tetra(m-hydroxyphenyl)chlorin (mTHPC), benzoporphyrin derivative mono acid ring (BPD-MA), zinc-phthalocyanin (ZnPC), protoporphyrin IX, chlorin e6, AlS4Pc, a texaphyrin, hypericin, or pheophorbide a.

15. The conjugate of claim 1, wherein the photosensitizer moieties are covalently attached to between from about 0.1% to about 80% of the available functionalities of the polymer.

16. The conjugate of claim 15, wherein the photosensitizer moieties are covalently attached to between from about 3% to about 50% of the available functionalities of the polymer.

17. The conjugate of claim 1, further comprising one or more quencher moieties conjugated to the polymer backbone.

18. The conjugate of claim 10, wherein the quencher moiety is in sufficient proximity to the photosensitizer to reduce the activity of the photosensitizer.

19. The conjugate of claim 17, wherein the quencher moiety comprises a non-fluorescing dye, DABCYL; DANSYL, QSY-7, a black hole quencher, a fluorophore, a nano-scaled semiconductor, a quantum dot, a nanotube, a fluorophore, or a gold nanoparticle.

20. The conjugate of claim 17, wherein the photosensitizers participate in energy transfer with the quencher.

21. The conjugate of claim 1, further comprising one or more biocompatibilizing units.

22. The conjugate of claim 21, wherein the biocompatibilizing unit is polyethyleneglycol (PEG), methoxypolyethyleneglycol (MPEG), polyethyleneglycol-diacid, PEG monoamine, MPEG monoamine, MPEG hydrazine, MPEG imidazole, methoxypropyleneglycol, a copolymer of polypropyleneglycol or methoxypropyleneglycol, dextran, polylactic-polyglycolic acid, 2-(N,N,N-Trimethylammonium)ethanoic acid, 1-methyl nicotinamide, 1-methyl nicotinamide, or monosuccinamide.

23. The conjugate of claim 1, further comprising one or more protecting units that reduces the rate of enzyme-activatable release of the photosensitizer.

24. The conjugate of claim 23, wherein the protecting unit comprises an amide, an imide, an imine, an ester, a thioester, a carbazone, a hydrazone, an oxime, an acetal, a ketal derivative of N-methylnicotinic acid, N-methylquinoline-X-carboxylic acid (where X=2, 3, 4, 5, 6, or 7), a substituted N-methylbenzoquinoline, a substituted N-methylacridine, a substituted N-methyl isoquinoline, a substituted N-methylphenanthredine or an N-alkylated derivative thereof, a substituted substituted pyridine, a benzopyridine, a dibenzopyridine, a dicarboxylic acid, oxalic acid, maleic acid, succinic acid, glutaiic acid, adipic acid, a polycarboxylic acid, citric acid, an amino acid, a peptide, an amino acid or peptide in which the amine functions are quaternized by methyl or other alkyl group, a sulfoacid, sulfoacetic acid, ascorbic acid-2-sulfate, an O-sulfonated amino acid, O-sulfo-serine, O-sulfo-tyrosine, O-sulfo-threonine, an O-sulfonated saccaride, a polysaccaride, a phosphorylated acid or amino acid, phosphogliceric acid, O-phospho-serine, O-phospho-threonine, O-phospho-tyrosine, ascorbic acid-2-phosphate, an O-phosphorylated saccaride or polysaccaride, glyceraldehyde-3-phosphate, glucose-6-phosphate, erythrose-4-phosphate, ribose-5-phosphate, pyridoxal-5-phosphate, or glusosamine-6-sulfate.

25. The conjugate of claim 1, further comprising a targeting moiety.

26. The conjugate of claim 25, wherein the targeting moiety comprises folic acid, a steroid such as cholesterol or a cholesterol ester, a cell adhesion molecule, a targeting peptide such as RGD, a saccharide, a polysaccharide, an oligonucleotide, an antibody, an antibody fragment or single chain antibody.

27. The conjugate of claim 1, wherein the molecular weight of the conjugate is between 1 kDa to 100,000 kDa.

28. The conjugate of claim 1, wherein the conjugate is comprised in a pharmaceutical composition.

29. The conjugate of claim 28, wherein the pharmaceutical composition is formulated for parenteral administration to a human.

30. A method of photochemotherapy comprising. administering the conjugate of any one of claims 1 to 29 to a subject in an effective amount.

31. The method of claim 30, wherein the subject is a human.

32. The method of claim 30, wherein the method comprises treating a disease.

33. The method of claim 32, wherein the disease is acne, a cell proliferative disease, a bacterial disease, a viral disease, a fungal infection, age-related macular degeneration, diabetic retinopathy, an arthritic disease, an inflammatory disease such as rheumatoid arthritis, or neovascularization.

34. The method of claim 33, wherein the cell proliferative disease is cancer, psoriasis, skin cancer, or actinic keratosis.

35. The method of claim 30, wherein the method is performed for a cosmetic purpose, such as hair removal or skin rejuvenation.

36. The method of claim 30, wherein the administration is topical or systemic.

37. The method of claim 30, wherein the method further comprises irradiation of part or all of the subject.

38. The method of claim 37, where the irradiation is carried out at a wavelength that is an absorption wavelength of the photosensitizer.

39. The method of claim 37, wherein the wavelength is between from about 350 to about 800nm.

40. The method of claim 39, wherein the wavelength is in the blue region, the red or near-infrared region, white light.

41. The method of claim 37, wherein the irradiation is carried out by a light source equipped with a filter.

42. The method of claim 37, wherein irradiation is performed with a laser.

43. The method of claim 37, wherein the step of said irradiation is performed within a time interval of 4 minutes to 168 hours after administration of the conjugate.

44. The method of claim 43, wherein the irradiation is performed within a time interval of 4 minutes to 72 hours after administration of the conjugate.

45. The method of claim 44, wherein the irradiation is performed within a time interval of 15 minutes to 48 hours after administration of the conjugate.

46. The method of claim 37, wherein the total fluence of light used for irradiation is between 2 J/cm² and 500 J/cm².
